(19) United States
(12) Patent Application Publication
(10) Pub. No.: US 2025/0136617 A1
(43) Pub. Date: May 1, 2025

(54) SMALL MOLECULES CXCR4 AGONISTS, METHOD OF SYNTHESIS, AND METHOD OF USE

(71) Applicants: The Regents of the University of Colorado, A Body Corporate, Boulder, CO (US); UNIVERSITY OF FLORIDA RESEARCH FOUNDATION, INCORPORATED, Gainesville, FL (US)

(72) Inventors: Kenneth Liechty, Denver, CO (US); Siobhan Malany, Gainesville, FL (US); Satyamaheshwar Peddibhotla, Gainesville, FL (US); Carlos Zgheib, Denver, CO (US)

(21) Appl. No.: 18/835,859

(22) PCT Filed: Feb. 7, 2023
(86) PCT No.: PCT/US2023/012490

§ 371 Date: Aug. 5, 2024

Related U.S. Application Data
(60) Provisional application No. 63/307,314, filed on Feb. 7, 2022.

(51) Int. Cl. C07D 513/04; A61K 31/549; A61P 17/02

(52) U.S. Cl. CPC C07D 513/04; A61K 31/549; A61P 17/02

(57) ABSTRACT

Small molecule CXC chemokine receptor type 4 (CXCR4) agonists are disclosed, methods of their manufacture, and uses thereof, in particular to improve healing and reduce the risk of injury for wound healing in diabetes.

### CROSS-REFERENCE TO RELATED APPLICATIONS

This application claims the benefit of priority of U.S. Provisional Patent Application No. 63/307,314, filed Feb. 7, 2022, the contents of which are incorporated herein by reference in their entirety for all purposes.

### STATEMENT REGARDING FEDERALLY SPONSORED RESEARCH OR DEVELOPMENT

This invention was made with government support under RO1 DK126371 and RO1 DK105010 awarded by the National Institutes of Health (NIH). The government has certain rights in the invention.

### TECHNICAL FIELD

This disclosure relates to small molecule CXCR4 agonists, methods of their manufacture, and uses thereof, in particular uses to improve healing and reduce the risk of injury for pathologic wound healing in diabetes.

### BACKGROUND

Diabetes produces a chronic inflammatory state that contributes to the development of vascular disease and impaired wound healing. Lower extremity ulcers are the leading cause of hospitalizations and amputations among diabetic patients. Despite the known individual and societal impacts of diabetic ulcers, there remains limited effective therapies that improve healing and reduce the risk of reinjury. There accordingly remains a need in the art for effective therapies that improve wound healing and reduce the risk of reinjury.

### SUMMARY

Described herein are compounds according to Formula I

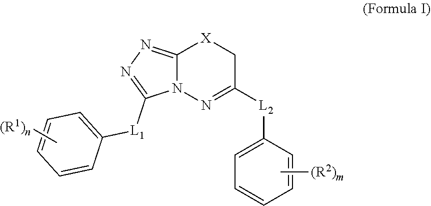

or a pharmaceutically acceptable salt thereof, wherein in Formula I,
   R¹ is each independently halogen, cyano, nitro, NR³R⁴, —C₁-C₆alkyl, —C₁-C₆haloalkyl, —C₂-C₆alkenyl, —C₂-C₆alkynyl, —C₁-C₆alkoxy, —C₀-C₆alkylCOOR⁵, —C₀-C₆alkyl(C₃-C₇cycloalkyl), —C₀-C₆alkyl(heterocycloalkyl), —C₀-C₆alkyl(aryl), or —C₀-C₆alkyl(heteroaryl), wherein groups except hydrogen, halogen, cyano, and nitro are optionally substituted with halogen, cyano, nitro, oxo (C═O), a —C₁-C₆alkyl, a —C₃-C₇cycloalkyl, a heterocycloalkyl, a heteroaryl, or an aryl;
   R² is each independently halogen, cyano, nitro, —C₁-C₆alkyl, —C₂-C₆alkenyl, —C₂-C₆alkynyl, —C₁-C₆alkoxy, —C₀-C₆alkylCOR⁵, —C₀-C₆alkylCOOR⁵, —C₀-C₆alkyl(C₃-C₇cycloalkyl), —C₀-C₆alkyl(heterocycloalkyl), —C₀-C₆alkyl(aryl), or —C₀-C₆alkyl(heteroaryl), —NR³C₀-C₆alkylCOR⁵, or NR³R⁴, wherein groups except hydrogen, halogen, cyano, and nitro are optionally substituted with halogen, cyano, nitro, oxo (C═O), a —C₁-C₆alkyl, —C₁-C₆haloalkyl, a —C₃-C₇cycloalkyl, a heterocycloalkyl, a heteroaryl, or an aryl, and wherein at least one R² is NR³R⁴ or —NR³C₀-C₆alkylCOR⁵;
   or wherein two R²s along with the C to which they are attached form a cyclic ring of 4 to 7 ring atoms or bicyclic ring of 8 to 10 ring atoms, the cyclic or bicyclic ring having 1, 2, or 3 ring atoms independently chosen from N, O, and S, wherein the cyclic or bicyclic ring is substituted with 0-2 substituents independently chosen from halogen, oxo, cyano, C₁-C₆alkyl, C₁-C₆alkoxy, C₁-C₆haloalkyl, and C₁-C₆haloalkoxy;
   X is CH₂, O, NR⁵, or S;
   L₁ and L₂ are each independently a bond (absent) or —C₁-C₂₀hydrocarbyl;
   R³ and R⁴ are each independently hydrogen, —C₁-C₆alkyl, —C₂-C₆alkenyl, —C₂-C₆alkynyl, —C₀-C₆alkyl-C₁-C₆alkoxy, —C₀-C₆alkylCOR⁶, —C₀-C₆alkyl-COOR⁶, —C₀-C₆alkyl-C₂-C₆alkenyl, C₀-C₆alkyl-C₂-C₆alkynyl, —C₀-C₆alkyl(aryl), or —C₀-C₆alkyl(heteroaryl), wherein groups except hydrogen are optionally substituted with halogen, cyano, nitro, oxo, —C₀-C₆alkylCOOR⁷, —C₁-C₆alkyl, —C₁-C₆haloalkyl, —C₃-C₇cycloalkyl, a heterocycloalkyl, a heteroaryl, or an aryl, and wherein at least one of R³ and R⁴ is not hydrogen;
   or R³ and R⁴ along with the N form a cyclic ring of 4 to 7 ring atoms or bicyclic heterocyclic ring of 8 to 10 ring atoms, the cyclic or bicyclic ring having 1, 2, or 3 ring atoms independently chosen from N, O, and S, wherein the cyclic or bicyclic heterocyclic ring is substituted with 0-2 substituents independently chosen from halogen, cyano, oxo, C₁-C₆alkyl, C₁-C₆alkoxy, C₁-C₆haloalkyl, and C₁-C₆haloalkoxy;
   R⁵, R⁶, and R⁷ are each independently hydrogen, —C₁-C₆alkyl, —C₁-C₆haloalkyl, —C₀-C₆alkyl(C₃-C₇cycloalkyl), —C₁-C₆alkyl-C₁-C₆alkoxy, —C₁-C₆alkyl-C₂-C₆alkenyl, or C₁-C₆alkyl-C₂-C₆alkynyl; and
   n and m are each independently an integer of 1, 2, 3, 4, or 5.

A method for the synthesis of the compounds of Formula I is further described.

A method of treating a patient in need of treatment with a CXCR4 agonist, the method comprising administering to the patient an effective amount of the compound of Formula I above or a compound of Formula II

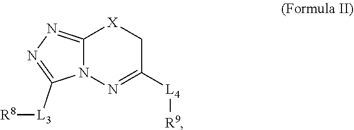

or a pharmaceutically acceptable salt thereof, wherein in Formula II,
X is CH₂, O, NR⁵, or S;
L₃ and L₄ are each independently a bond (absent) or —C₁-C₂₀hydrocarbyl;
R⁸ and R⁹ are each independently —C₃-C₇cycloalkyl, a heterocycloalkyl, a heteroaryl, or an aryl, each R⁸ and R⁹ is optionally substituted with halogen, cyano, nitro, oxo (C═O), —C₁-C₆alkyl, —C₂-C₆alkenyl, —C₂-C₆alkynyl, —C₁-C₆alkoxy, —C₀-C₆alkylCOR¹⁰, —C₀-C₆alkylCOOR¹⁰, —C₀-C₆alkyl(C₃-C₇cycloalkyl), —C₀-C₆alkyl(heterocycloalkyl), —C₀-C₆alkyl(aryl), or —C₀-C₆alkyl(heteroaryl), —NR¹⁰C₀-C₆alkylCOR¹¹, or NR¹⁰R¹¹, wherein groups except hydrogen, halogen, cyano, and nitro are optionally substituted with halogen, cyano, nitro, oxo, —C₁-C₆alkyl, —C₁-C₆haloalkyl, —C₃-C₇cycloalkyl, a heterocycloalkyl, a heteroaryl, or an aryl;
R¹⁰ and R¹¹ are each independently hydrogen, —C₁-C₆alkyl, —C₂-C₆alkenyl, —C₂-C₆alkynyl, —C₀-C₆alkyl-C₁-C₆alkoxy, —C₀-C₆alkylCOR¹², —C₀-C₆alkyl-COOR¹², —C₀-C₆alkyl-C₂-C₆alkenyl, C₀-C₆alkyl-C₂-C₆alkynyl, —C₀-C₆alkyl(aryl), or —C₀-C₆alkyl(heteroaryl), wherein groups except hydrogen are optionally substituted with halogen, cyano, nitro, oxo, —C₀-C₆alkylCOOR¹², —C₁-C₆alkyl, —C₁-C₆haloalkyl, —C₃-C₇cycloalkyl, a heterocycloalkyl, a heteroaryl, or an aryl;
or for NR¹⁰R¹¹, R¹⁰ and R¹¹ along with the N form a cyclic ring of 4 to 7 ring atoms or bicyclic heterocyclic ring of 8 to 10 ring atoms, the cyclic or bicyclic ring having 1, 2, or 3 ring atoms independently chosen from N, O, and S, wherein the cyclic or bicyclic heterocyclic ring is substituted with 0-2 substituents independently chosen from halogen, cyano, oxo, —C₁-C₆alkyl, —C₁-C₆alkoxy, —C₁-C₆haloalkyl, and —C₁-C₆haloalkoxy; and
R⁵ and R¹² are each independently hydrogen, —C₁-C₆alkyl, —C₁-C₆haloalkyl, —C₀-C₆alkyl(C₃-C₇cycloalkyl), —C₁-C₆alkyl-C₁-C₆alkoxy, —C₁-C₆alkyl-C₂-C₆alkenyl, or —C₁-C₆alkyl-C₂-C₆alkynyl.
   A method of treating a patient in need of treatment with a CXCR4 agonist, the method comprising administering to the patient an effective amount of the compound of Formula III

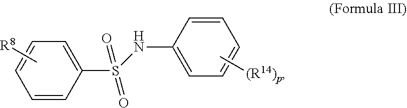

or a pharmaceutically acceptable salt thereof, wherein in Formula III,
R⁸ is —C₃-C₇cycloalkyl, a heterocycloalkyl, a heteroaryl, or an aryl, wherein R⁸ is optionally substituted with halogen, cyano, nitro, oxo (C═O), —C₁-C₆alkyl, —C₂-C₆alkenyl, —C₂-C₆alkynyl, —C₁-C₆alkoxy, —C₀-C₆alkylCOR¹⁰, —C₀-C₆alkylCOOR¹⁰, —C₀-C₆alkyl(C₃-C₇cycloalkyl), —C₀-C₆alkyl(heterocycloalkyl), —C₀-C₆alkyl(aryl), or —C₀-C₆alkyl(heteroaryl), —NR¹⁰C₀-C₆alkylCOR¹¹, or NR¹⁰R¹¹, wherein groups except hydrogen, halogen, cyano, and nitro are optionally substituted with halogen, cyano, nitro, oxo, —C₁-C₆alkyl, —C₁-C₆haloalkyl, —C₃-C₇cycloalkyl, a heterocycloalkyl, a heteroaryl, or an aryl;
R¹⁴ is hydrogen, halogen, cyano, nitro, —C₁-C₆alkyl, —C₂-C₆alkenyl, —C₂-C₆alkynyl, —C₁-C₆alkoxy, —C₀-C₆alkylCOR¹⁵, —C₀-C₆alkylCOOR¹⁵, —C₀-C₆alkyl(C₃-C₇cycloalkyl), —C₀-C₆alkyl(heterocycloalkyl), —C₀-C₆alkyl(aryl), or —C₀-C₆alkyl(heteroaryl), —NR¹⁶C₀-C₆alkylCOR¹⁵, or NR¹⁶R¹⁷, wherein groups except hydrogen, halogen, cyano, and nitro are optionally substituted with halogen, cyano, nitro, oxo, —C₁-C₆alkyl, —C₁-C₆haloalkyl, —C₃-C₇cycloalkyl, a heterocycloalkyl, a heteroaryl, or an aryl;
or wherein two R¹⁴s along with the C to which they are attached form a cyclic ring of 4 to 7 ring atoms or bicyclic ring of 8 to 10 ring atoms, the cyclic or bicyclic ring having 1, 2, or 3 ring atoms independently chosen from N, O, and S, wherein the cyclic or bicyclic ring is substituted with 0-2 substituents independently chosen from halogen, oxo, cyano, —C₁-C₆alkyl, —C₁-C₆alkoxy, —C₁-C₆haloalkyl, and —C₁-C₆haloalkoxy;
R¹⁰ and R¹¹ are each independently hydrogen, —C₁-C₆alkyl, —C₂-C₆alkenyl, —C₂-C₆alkynyl, —C₀-C₆alkyl-C₁-C₆alkoxy, —C₀-C₆alkylCOR¹², —C₀-C₆alkyl-COOR¹², —C₀-C₆alkyl-C₂-C₆alkenyl, C₀-C₆alkyl-C₂-C₆alkynyl, —C₀-C₆alkyl(aryl), or —C₀-C₆alkyl(heteroaryl), wherein groups except hydrogen are optionally substituted with halogen, cyano, nitro, oxo, —C₀-C₆alkylCOOR¹², —C₁-C₆alkyl, —C₁-C₆haloalkyl, —C₃-C₇cycloalkyl, a heterocycloalkyl, a heteroaryl, or an aryl;
or for NR¹⁰R¹¹, R¹⁰ and R¹¹ along with the N form a cyclic ring of 4 to 7 ring atoms or bicyclic heterocyclic ring of 8 to 10 ring atoms, the cyclic or bicyclic ring having 1, 2, or 3 ring atoms independently chosen from N, O, and S, wherein the cyclic or bicyclic heterocyclic ring is substituted with 0-2 substituents independently chosen from halogen, cyano, oxo, —C₁-C₆alkyl, —C₁-C₆alkoxy, —C₁-C₆haloalkyl, and —C₁-C₆haloalkoxy;
R¹⁶ and R¹⁷ are each independently hydrogen, —C₁-C₆alkyl, —C₂-C₆alkenyl, —C₂-C₆alkynyl, —C₀-C₆alkyl-C₁-C₆alkoxy, —C₀-C₆alkylCOR¹⁸, —C₀-C₆alkyl-COOR¹⁸, —C₀-C₆alkyl-C₂-C₆alkenyl, C₀-C₆alkyl-C₂-C₆alkynyl, —C₀-C₆alkyl(aryl), or —C₀-C₆alkyl(heteroaryl), wherein groups except hydrogen are optionally substituted with halogen, cyano, nitro, oxo, —C₀-C₆alkylCOOR¹⁸, —C₁-C₆alkyl, —C₁-C₆haloalkyl, —C₃-C₇cycloalkyl, a heterocycloalkyl, a heteroaryl, or an aryl;
or R¹⁶ and R¹⁷ along with the N form a cyclic or bicyclic heterocyclic ring of 8 to 10 ring atoms, having 1, 2, or 3 ring atoms independently chosen from N, O, and S, wherein the cyclic or bicyclic heterocyclic ring is substituted with 0-2 substituents independently chosen from halogen, cyano, oxo, —C₁-C₆alkyl, —C₁-C₆alkoxy, —C₁-C₆haloalkyl, and —C₁-C₆haloalkoxy;
R¹⁵ and R¹⁸ are each independently is hydrogen, —C₁-C₆alkyl, —C₁-C₆haloalkyl, —C₀-C₆alkyl(C₃-C₇cycloalkyl), —C₁-C₆alkyl-C₁-C₆alkoxy, —C₁-C₆alkyl-C₂-C₆alkenyl, or —C₁-C₆alkyl-C₂-C₆alkynyl; and
p is 1,2,3,4, or 5.

### BRIEF DESCRIPTION OF DRAWINGS

FIGS. 1A and 1B Dose-dependent b-arrestin recruitment in U2OS-CXCR4bla cells of SDF-1α purchased from 1A) R&D Systems (Log EC50=−7.33±0.06) and 1B) Peprotech (Log EC50=−7.78±0.05).

FIG. 2A, 2B, 2C, and 2D illustrate that UCUF-728 is a positive modulator of β-arrestin signaling and cell migration. FIG. 2A) UCUF-728 (open circles) shows dose-dependent partial activation of β-arrestin recruitment in CXCR4-bla U2OS Cells compared to SDF-1α dose-dependent response (closed circles). Error bars are+/−SD of triplicate determinations. FIG. 2B) UCUF-728 (open circles) does not show dose-dependent activity in cAMP signaling in CHO-K1-CXCR4. SDF-1α dose-dependent response of cAMP signaling is shown (closed circles) and AMD3100 dose-dependent inhibition of 40 nM SDF-1α is also shown (closed squares). Error bars are+/−SD of triplicate determinations. FIG. 2C) UCUF-728 (open circles) shows dose-dependent partial activation of CEM-CCRF cell migration. FIG. 2D) SDF-1a shows typical bell-shaped curve in CEM-CCRF cell migration (closed circles) and this response is modulated in the presence of 1 μM (closed squares) and 10 μM (open circles) UCUF-728. Data in C) and D) are representative experiments of two independent experiments.

FIG. 3 shows Percent Migration of Lymphoblasts Antagonized by AMD 3100. AMD3100 dose-dependently inhibited migration in CEM lymphoblasts with logIC50=−7.5±0.6. Data is normalized to media only (low control) and 6 nM SDF-1a (high control). Data is average of duplicate experiments performed in triplicate.

FIG. 4 shows Antagonism of SDF-1a binding to CXCR4 Receptor. SDF-1a binding was validated using the Tag-Lite CXCR4 system (CisBio) according to manufacturer protocol. AMD3100 dose-dependently displaced SDF-1a saturation binding (closed circles) and UCUF-728 did not displace SDF-1a saturation binding (open circles).

FIG. 5 shows realtime qPCR (mean±SD, n=3 per group) analysis of miR-15b gene expression relative to internal control U6 in human diabetic dermal fibroblasts (“Db”) and non-diabetic dermal fibroblasts (“non-Db”) with UCUF 728 treated at dose of 0, 0.1, 1 and 10 uM.

FIG. 6 shows realtime qPCR (mean±SD, n=3 per group) analysis of miR-29a gene expression relative to internal control U6 in human diabetic dermal fibroblasts (“Db”) and non-diabetic dermal fibroblasts (“non-Db”) with UCUF 728 treated at dose of 0, 0.1, 1 and 10 uM.

FIG. 7 shows wound size change during the healing process of initial 8 mm wounds in Db mice treated by dermal injection of 10 uM UCUF-728 or PBS, n=5 per group. * p<0.05 or ** p<0.01 comparing between PBS treated group to 100 uM UCUF-728 treated group.

FIG. 8A) CD31+ staining of representative sections of diabetic wounds after 7 days of treatment with control PBS (n=5) or 100 uM UCUF-728 (n=5). FIG. 8B) Quantitative analysis of number of vessels (CD31 staining) per 20× field. Comparison was performed between control PBS and 100 uM UCUF-728 treated wounds.

FIG. 9A, 9B, 9C, and 9D. UCUF-965 is a partial allosteric activator of CXCR4 cell signaling FIG. 9A) UCUF-965 (open squares) shows dose-dependent partial activation of β-arrestin recruitment in CXCR4-bla U2OS cells compared to SDF-1α (closed circles). Additionally, SDF-1α titration in the presence of 1 μM 965 (open triangles) is not inhibited in β-arrestin recruitment. FIG. 9B) In CXCR4 cAMP CHO cells SDF-1α (closed circles) is shown for full cAMP inhibition agonist response reference. AMD 3100 antagonist at 1 μM (open diamonds) reduces the activity of SDF-1α to inhibit cAMP and UCUF-965 (open squares) shows dose-dependent partial inhibition of forskolin-stimulated cAMP production compared to SDF-1α maximal response (closed circles). FIG. 9C) Simultaneous addition of UCUF965 potentiates the cAMP response of SDF-1α in a dose dependent manner ranging from 100 nM (closed squares), 1 μM (open triangles) and 5 μM (open squares) indicated by the left-shift of the dose response curve and EC₅₀ value and higher Eₘₐₓ compared to SDF-1α treatment alone (closed circles). FIG. 9D) UCUF-965 did not displace SDF-1α saturation binding in Tag-Lite CXCR4 cells (closed squares). AMD3100 dose-dependently inhibited SDF-1α saturation binding (open diamonds). Data is average of triplicate biological determinations tested in duplicate.

FIG. 10A, 10B, and 10C. UCUF-965 activates endogenous CXCR4 cell signaling in CEM-CCRF lymphoblasts FIG. 10A) In CEM-CCRF cells, small molecule antagonist AMD 3100 at 5 μM (closed diamonds) is able to inhibit the migration response induced by SDF-1α (closed circles). UCUF-965 (open squares) is a partial activator of CXCR4-mediated cell migration compared to full agonist SDF-1α (closed circles). Additionally, AMD3100 at 5 μM (open diamonds) does not inhibit the migratory response of CEM-CCRF cells induced by UCUF-965. FIG. 10B) Calcium flux induced by 10 nM SDF-1α (closed circles) is inhibited in a dose-dependent manner by pre-treatment with known CXCR4 inhibitor, AMD 3100. Similarly, calcium flux induced by 10 μM UCUF-965 (open squares) is dose-dependently inhibited by pre-treatment with AMD 3100. Error bars are+/−SD of triplicate determination. FIG. 10C) In CEM-CCRF cells, 10 nM SDF-1α (solid black line) and 10 μM UCUF-965 (solid gray line) induce CXCR4-mediated calcium flux. Dotted lines indicate standard deviations of triplicate biological determinations.

FIG. 11 shows miR-29a expression level was decreased with UCUF-965 treatment in Human dermal fibroblast.

FIG. 12 shows miR-15b expression level was decreased with UCUF-965 treatment in Human dermal fibroblast.

FIG. 13 shows wound size change during the healing process of initial 8 mm wounds in Db mice treated by dermal injection of 10 μM UCUF-965 or PBS, n=5 per group.

### DETAILED DESCRIPTION

Small molecule CXC chemokine receptor type 4 (CXCR4) agonists are disclosed, also disclosed are the methods of their manufacture, and uses thereof, in particular use to improve healing and reduce the risk of injury for pathologic wound healing in diabetes.

Diabetes has reached epidemic proportions in the United States and globally, and impaired diabetic wound healing is a significant and growing clinical problem. Despite the enormous impact of these wounds on both individuals and society, effective therapies are lacking. Improving/accelerating the healing of cutaneous wound in diabetics has the potential to significantly improve patient outcomes and reduce healthcare expenditures. There is an unmet need to develop small molecule therapeutics to effectively promote healing of diabetic wounds.

Normal wound repair follows an orderly and well-defined sequence of events that requires the interaction of many cell types, such as inflammatory cells, fibroblasts, keratinocytes, endothelial cells and progenitor cells, as well as the involvement of many growth factors, extracellular matrix (ECM) proteins, and enzymes. In diabetic wound healing, this complex orchestration of wound healing processes is disrupted. This impairment is associated with significantly decreased production of granulation tissue and an increased epithelial gap, compared to non-diabetic wounds. The production of granulation tissue is dependent on the formation of new vessels in the wound bed, synthesis of extracellular matrix, and provides the substrate for epithelial cell migration and subsequent wound closure. Stromal cell-derived factor 1α (SDF-1α or CXCL12) is a CXC chemokine that functions via activation of the CXC chemokine receptor type 4 (CXCR4) receptor to recruit hematopoietic cells to locations of tissue injury and promote tissue repair. SDF-1α is a potent chemokine involved in progenitor cell recruitment, angiogenesis, and granulation tissue formation, mediated through binding to the CXCR4 receptor and the establishment of a chemotactic gradient. SDF-1α secretion has also been shown to be upregulated by hypoxia inducible factor (HIF-1α) and its expression is increased in areas of tissue injury. These observations have led to suggestions that SDF-1α may also have a central role in directing cells to injured tissues to facilitate tissue repair. Expression of SDF-1α is reduced in diabetic wounds, suggesting a potential contribution to wound healing impairment and presenting the CXCR4 receptor as a target for therapeutic investigations.

It has been found that diabetic wounds produced significantly less SDF-1α both at mRNA and protein level compared to normal wound tissue. Is has been shown that local application of Mesenchymal Stem Cells (MSCs) enhances wound closure in a diabetic mouse model. MSC-treated wounds had decreased inflammation response, decreased epithelial gap, and increased vessel density. Furthermore, this effect appears to be due, in part, to increased production of SDF-1α in wounds.

Analysis of diabetic wounds produced significantly less SDF-1α both in mRNA, and protein level. In a gain of function experiment, SDF-1α was overexpressed by lentivirus vector, and SDF-1α treatment resulted in greater granulation tissue, smaller epithelial gap, and smaller wound size in diabetic wounds. Expression of a mutant form of SDF-1α competitively inhibits activation of CXCR4 by endogenous SDF-1α significantly increases inflammation, decreases angiogenesis and impairs the rate of wound healing in the diabetic mouse. Small molecule orthosteric or allosteric activators of endogenous SDF-1α binding and CXCR4 signaling hold tremendous therapeutic promise in diabetic wound healing, especially if developed as topical therapy without associated systemic activation. Small molecule therapeutics are also desirable due to the proteolytic nature of wounds throughout the healing process which limit application of peptide therapeutics and other biomolecules.

In a loss of function experiment, a lentiviral vector that expresses a mutant form of SDF-1α that binds, but does not activate, CXC chemokine receptor type 4 (CXCR4) was injected and its effect on granulation tissue formation, angiogenesis, inflammation, cell migration, and wound healing was measured. It was found that competitive inhibition of SDF-1α significantly impairs the rate of wound healing, decreases angiogenesis, and increases inflammation in the diabetic mouse. Competitive inhibition of SDF-1α significantly impairs the rate of wound healing, decreases angiogenesis, and increases inflammation in the diabetic mouse migration and subsequent wound closure. This data indicated that SDF-1α is a key factor in the wound-healing process that could be targeted to correct the diabetic wound-healing defect. Discovery of small molecule positive modulators that can activate CXCR4 receptor and its downstream signaling pathway, thereby, blocking SDF-1α response, will provide a novel topical therapy for diabetic wound healing with reduced risk for systemic receptor activation, and has great potential for clinical application.

Small molecules that are CXCR4 agonists as novel therapies for promoting wound healing and tissue repair in diabetes are disclosed herein, including compounds of Formula I, Formula Ia, Formula Ib, Formula II, Formula III, a subformula thereof, and pharmaceutically acceptable salts thereof. Not wishing to be bound by theory, but it is hypothesized that the small molecule CXCR4 agonists will improve diabetic wound healing by correcting abnormal migration, angiogenesis, and the associated dysregulated microRNA.

The disclosure includes the following particular embodiments, thiadiazine compounds of Formula I

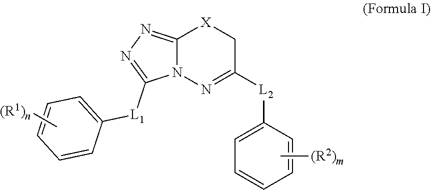

or a pharmaceutically acceptable salt thereof, wherein in Formula I,
   R¹ is each independently halogen, cyano, nitro, NR³R⁴, —C₁-C₆alkyl, —C₁-C₆haloalkyl, —C₂-C₆alkenyl, —C₂-C₆alkynyl, —C₁-C₆alkoxy, —C₀-C₆alkylCOOR⁵, —C₀-C₆alkyl(C₃-C₇cycloalkyl), —C₀-C₆alkyl(heterocycloalkyl), —C₀-C₆alkyl(aryl), or —C₀-C₆alkyl(heteroaryl), wherein groups except hydrogen, halogen, cyano, and nitro are optionally substituted with halogen, cyano, nitro, oxo (C═O), a —C₁-C₆alkyl, a —C₃-C₇cycloalkyl, a heterocycloalkyl, a heteroaryl, or an aryl;
   R² is each independently halogen, cyano, nitro, —C₁-C₆alkyl, —C₂-C₆alkenyl, —C₂-C₆alkynyl, —C₁-C₆alkoxy, —C₀-C₆alkylCOR⁵, —C₀-C₆alkylCOOR⁵, —C₀-C₆alkyl(C₃-C₇cycloalkyl), —C₀-C₆alkyl(heterocycloalkyl), —C₀-C₆alkyl(aryl), or —C₀-C₆alkyl(heteroaryl), —NR³C₀-C₆alkylCOR⁵, or NR³R⁴, wherein groups except hydrogen, halogen, cyano, and nitro are optionally substituted with halogen, cyano, nitro, oxo (C═O), a —C₁-C₆alkyl, —C₁-C₆haloalkyl, a —C₃-C₇cycloalkyl, a heterocycloalkyl, a heteroaryl, or an aryl, and wherein at least one R² is NR³R⁴ or —NR³C₀-C₆alkylCOR⁵;
   or wherein two R²s along with the C to which they are attached form a cyclic ring of 4 to 7 ring atoms or bicyclic ring of 8 to 10 ring atoms, the cyclic or bicyclic ring having 1, 2, or 3 ring atoms independently chosen from N, O, and S, wherein the cyclic or bicyclic ring is substituted with 0-2 substituents independently chosen from halogen, oxo, cyano, C₁-C₆alkyl, C₁-C₆alkoxy, C₁-C₆haloalkyl, and C₁-C₆haloalkoxy;
   X is CH₂, O, NR⁵, or S;
   L₁ and L₂ are each independently a bond (absent) or —C₁-C₂₀hydrocarbyl;
   R³ and R⁴ are each independently hydrogen, —C₁-C₆alkyl, —C₂-C₆alkenyl, —C₂-C₆alkynyl, —C₀-C₆alkyl-C₁-C₆alkoxy, —C₀-C₆alkylCOR⁶, —C₀-C₆alkyl-COOR⁶, —C₀-C₆alkyl-C₂-C₆alkenyl, C₀-C₆alkyl-C₂-C₆alkynyl, —C₀-C₆alkyl(aryl), or —C₀-C₆alkyl(heteroaryl), wherein groups except hydrogen are optionally substituted with halogen, cyano, nitro, oxo, —C₀-C₆alkylCOOR⁷, —C₁-C₆alkyl, —C₁-C₆haloalkyl, —C₃-C₇cycloalkyl, a heterocycloalkyl, a heteroaryl, or an aryl, and wherein at least one of R³ and R⁴ is not hydrogen;
   or R³ and R⁴ along with the N form a cyclic ring of 4 to 7 ring atoms or bicyclic heterocyclic ring of 8 to 10 ring atoms, the cyclic or bicyclic ring having 1, 2, or 3 ring atoms independently chosen from N, O, and S, wherein the cyclic or bicyclic heterocyclic ring is substituted with 0-2 substituents independently chosen from halogen, cyano, oxo, C₁-C₆alkyl, C₁-C₆alkoxy, C₁-C₆haloalkyl, and C₁-C₆haloalkoxy;
   R⁵, R⁶, and R⁷ are each independently hydrogen, —C₁-C₆alkyl, —C₁-C₆haloalkyl, —C₀-C₆alkyl(C₃-C₇cycloalkyl), —C₁-C₆alkyl-C₁-C₆alkoxy, —C₁-C₆alkyl-C₂-C₆alkenyl, or C₁-C₆alkyl-C₂-C₆alkynyl; and
   n and m are each independently an integer of 1, 2, 3, 4, or 5.

In some embodiments, a compound of Formula I, wherein
R¹ is —C₁-C₆alkoxy,
R² is NR³R⁴;
R³ and R⁴ are each independently hydrogen, —C₁-C₆alkyl-C₁-C₆alkoxy, —C₁-C₆alkyl-COOR⁵, or C₁-C₆alkyl-C₂-C₆alkynyl;
X is S;
L₁ and L₂ are each a bond; and
n and m are each independently is an integer of 1, 2, 3, 4, or 5.

In some embodiments the compound of Formula I is a compound of Formula Ia,

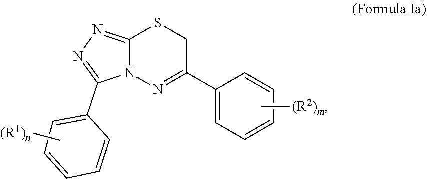

or a pharmaceutically acceptable salt thereof, wherein R¹, R², n, and m are as previously defined.

In some embodiments, a compound of Formula Ia, wherein
R¹ is —C₁-C₆alkoxy,
R² is NR³R⁴;
R³ and R⁴ are each independently hydrogen, —C₁-C₆alkyl-C₁-C₆alkoxy, —C₁-C₆alkyl-COOR⁵, or C₁-C₆alkyl-C₂-C₆alkynyl; and
n and m are each independently is an integer of 1, 2, or 3.

In some embodiments, the compound of Formula I is a compound of Formula Ib,

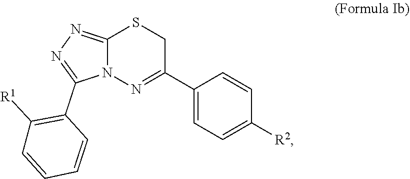

or a pharmaceutically acceptable salt thereof wherein R¹ and R² are as previously defined.

In some embodiments, a compound of Formula Ib, wherein
R¹ is —C₁-C₆alkoxy,
R² is NR³R⁴; and
   R³ and R⁴ are each independently hydrogen, —C₁-C₆alkyl-C₁-C₆alkoxy, —C₁-C₆alkyl-COOR⁵, or C₁-C₆alkyl-C₂-C₆alkynyl.

In some other embodiments, the compound of Formula I is,

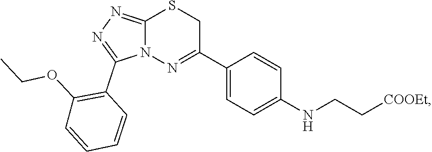 [SMILES: CCOC(=O)CCNc1ccc(C2=Nn3c(nnc3-c3ccccc3OCC)SC2)cc1]

or a pharmaceutically acceptable salt thereof.

In another embodiment, the compound of Formula I is,

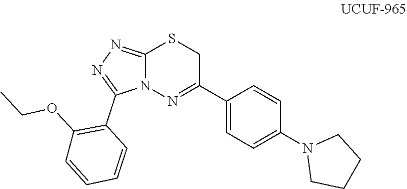 [SMILES: CCOc1ccccc1-c1nnc2n1N=C(c1ccc(N3CCCC3)cc1)CS2]

or a pharmaceutically acceptable salt thereof.

To determine the mode of action of UCUF-965, the compound was tested in the presence of increasing concentrations of SDF-1α. UCUF-965 did not inhibit the action of SDF-1α in the β-arrestin and cAMP signaling assays typical of a partial agonist binding orthosterically in the presence of a full agonist. UCUF-965 did not competitively displace fluorescently labeled SDF-1α binding to CXCR4 expressing cells, indicating non-orthosteric binding and mode of action. Moreover, it was found that pretreatment with UCUF-965 potentiated SDF-1α-mediated inhibition of cAMP production upon stimulation by forskolin (Gᵢ signaling) by lowering the EC₅₀ (left-shift) and doubling the Eₘₐₓ compared to treatment by SDF-1α alone. This result indicates allosteric activation of CXCR4/CXCL12 signaling by UCUF-965. AMD3100 dose-dependently inhibited SDF-1α-and UCUF-965 mediated calcium flux with IC₅₀ values of 60 nM and 640 nM, respectively, indicating UCUF-965 is a weaker activator compared to the natural ligand. The data collectively supports that UCUF-965 interacts with the CXCR4 receptor and activates CXCL12 mediated signaling as a positive allosteric modulator.

The functional activity of UCUF-965 was assessed in a transwell migration assay utilizing CEM-CCRF human lymphoblast cells and found that UCUF-965 induced migration with 10-fold lower potency compared to its activity in the β-arrestin recruitment assay and AMD3100 was unable to inhibit UCUF-965-induced migration consistent with the allosteric mode of action proposed for UCUF-965. Treatment of murine diabetic fibroblasts with UCUF-965 resulted in the induction of miR146a and suppression of miR15b and miR29a expression, with response observed even at the lowest concentration tested (0.1 μM). MiR-15b is a negative modulator of angiogenesis and is upregulated in diabetic wounds during the early phase of healing. This results in decreased expression of pro-angiogenic target genes, including vascular endothelial growth factor (VEGF), hypoxia inducible factor (HIF-1), and B-cell lymphoma 2 (BCL2). MiR-29a is upregulated in diabetic wounds which potentially leads to decreased collagen I content in diabetic wounds and delayed healing. MiR146a inhibits inflammation and is downregulated in diabetic wounds. UCUF-965 may contribute towards accelerated wound closure by modulation of miR-15b, miR29a and miR146a expression in diabetic wounds among others. In order to test this hypothesis, the efficacy of UCUF-965 was evaluated in accelerating diabetic wound healing in a mouse model. Murine dorsal wounds were allowed to heal completely with pictures taken daily to measure wound size until full closure. Diabetic wounds treated with PBS healed at day 22, while wounds treated with 10 μM UCUF-965 healed 36% faster at day 14. The preliminary study demonstrated that UCUF-965, a positive allosteric modulator (PAM) of CXCR4/CXCL12 signaling can significantly reduce wound healing in diabetic mice model.

In an aspect, described is a process of synthesizing the compound of Formula I, the process including: condensation, e.g. under thermal heating or microwave irradiation, by reacting a 4-amino-4H-1,2,4-triazole-3-thiol intermediate A with intermediate B wherein “LG” is a leaving group to afford the compound of Formula I, where X is S, according to Scheme I.

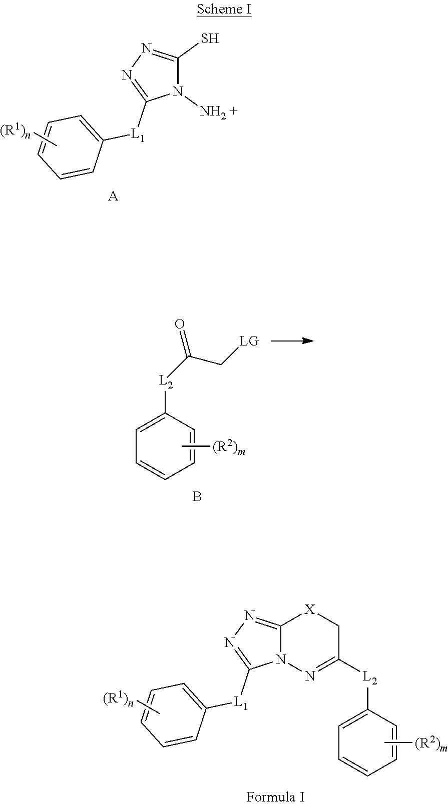

In another aspect, a process of synthesizing the compound of Formula I includes the reaction of intermediate C with an appropriate aldehyde, ketone, acetal or equivalent and subsequent reduction of the imine with sodium triacetoxyborohydride or other hydride reagent according to Scheme II.

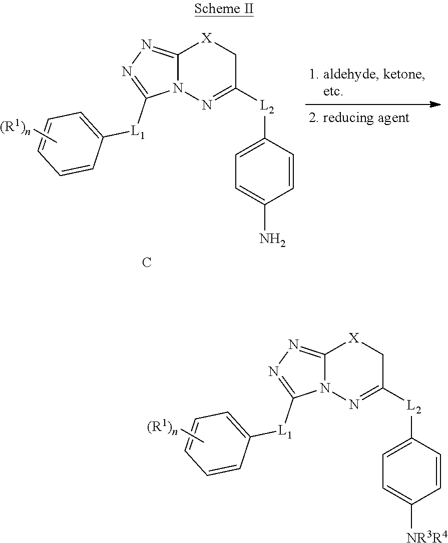

### Treatment Methods

Activation of CXCR4 receptor signaling with partial agonists or positive allosteric modulators (PAMs) provides a potential for small molecule therapeutic discovery and development. Allosteric modulators targeting GPCRs such as CXCR4 act at sites separate from the orthosteric ligand binding sites to modulate endogenous ligand activity. Since they do not compete with the natural ligand, their effect may be saturable upon occupation of all allosteric sites on the target. By preserving endogenous receptor-ligand signaling, allosteric positive modulators may potentially offer improved selectivity and safety over orthosteric agonists. This can be important for receptor sub-types that have a common ligand, share conserved sequences around the ligand binding orthosteric site, form heterodimers with other chemokine receptors and regulate normal physiology. In addition, allosteric modulators offer unique modes of action that control only selective functions of a receptor, expanding the range of therapeutic utility.

In another aspect, described is a method of treating a patient in need of treatment with a CXCR4 agonist, the method comprising,
administering to the patient an effective amount of the compound of Formula II

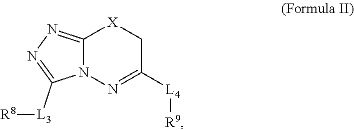

or a pharmaceutically acceptable salt thereof, wherein in Formula II,
   X is CH₂, O, NR²⁰, or S;
   L₃ and L₄ are each independently a bond (absent) or —C₁-C₂₀hydrocarbyl;
   R⁸ and R⁹ are each independently —C₃-C₇cycloalkyl, a heterocycloalkyl, a heteroaryl, or an aryl, each R⁸ and R⁹ is optionally substituted with halogen, cyano, nitro, oxo (C═O), —C₁-C₆alkyl, —C₂-C₆alkenyl, —C₂-C₆alkynyl, —C₁-C₆alkoxy, —C₀-C₆alkylCOR¹⁰, —C₀-C₆alkylCOOR¹⁰, —C₀-C₆alkyl(C₃-C₇cycloalkyl), —C₀-C₆alkyl(heterocycloalkyl), —C₀-C₆alkyl(aryl), or —C₀-C₆alkyl(heteroaryl), —NR¹⁰C₀-C₆alkylCOR¹¹, or NR¹⁰R¹¹, wherein groups except hydrogen, halogen, cyano, and nitro are optionally substituted with halogen, cyano, nitro, oxo, —C₁-C₆alkyl, —C₁-C₆haloalkyl, —C₃-C₇cycloalkyl, a heterocycloalkyl, a heteroaryl, or an aryl;
   R¹⁰ and R¹¹ are each independently hydrogen, —C₁-C₆alkyl, —C₂-C₆alkenyl, —C₂-C₆alkynyl, —C₀-C₆alkyl-C₁-C₆alkoxy, —C₀-C₆alkylCOR¹², —C₀-C₆alkyl-COOR¹², —C₀-C₆alkyl-C₂-C₆alkenyl, C₀-C₆alkyl-C₂-C₆alkynyl, —C₀-C₆alkyl(aryl), or —C₀-C₆alkyl(heteroaryl), wherein groups except hydrogen are optionally substituted with halogen, cyano, nitro, oxo, —C₀-C₆alkylCOOR¹², —C₁-C₆alkyl, —C₁-C₆haloalkyl, —C₃-C₇cycloalkyl, a heterocycloalkyl, a heteroaryl, or an aryl;
   or for NR¹⁰R¹¹, R¹⁰ and R¹¹ along with the N form a cyclic ring of 4 to 7 ring atoms or bicyclic heterocyclic ring of 8 to 10 ring atoms, the cyclic or bicyclic ring having 1, 2, or 3 ring atoms independently chosen from N, O, and S, wherein the cyclic or bicyclic heterocyclic ring is substituted with 0-2 substituents independently chosen from halogen, cyano, oxo, —C₁-C₆alkyl, —C₁-C₆alkoxy, —C₁-C₆haloalkyl, and —C₁-C₆haloalkoxy; and
R²⁰ and R¹² are each independently hydrogen, —C₁-C₆alkyl, —C₁-C₆haloalkyl, —C₀-C₆alkyl(C₃-C₇cycloalkyl), —C₁-C₆alkyl-C₁-C₆alkoxy, —C₁-C₆alkyl-C₂-C₆alkenyl, or —C₁-C₆alkyl-C₂-C₆alkynyl. The method of treating a patient in need of treatment with a CXCR4 agonist includes treating a wound and promoting healing of a wound, specifically treating a diabetic wound and promoting the healing of a diabetic wound.

In another aspect, the compound of Formula II is a compound of Formula IIa,

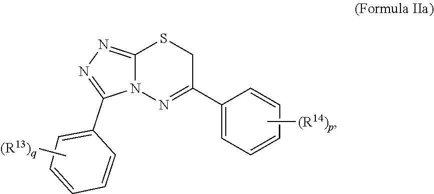

or a pharmaceutically acceptable salt thereof, wherein in Formula IIa,
R¹³ and R¹⁴ are each independently hydrogen, halogen, cyano, nitro, —C₁-C₆alkyl, —C₂-C₆alkenyl, —C₂-C₆alkynyl, —C₁-C₆alkoxy, —C₀-C₆alkylCOR¹⁵, —C₀-C₆alkylCOOR¹⁵, —C₀-C₆alkyl(C₃-C₇cycloalkyl), —C₀-C₆alkyl(heterocycloalkyl), —C₀-C₆alkyl(aryl), or —C₀-C₆alkyl(heteroaryl), —NR¹⁶C₀-C₆alkylCOR¹¹, or NR¹⁶R¹⁷, wherein groups except hydrogen, halogen, cyano, and nitro are optionally substituted with halogen, cyano, nitro, oxo, —C₁-C₆alkyl, —C₁-C₆haloalkyl, —C₃-C₇cycloalkyl, a heterocycloalkyl, a heteroaryl, or an aryl;
or wherein two R¹⁴s along with the C to which they are attached form a cyclic ring of 4 to 7 ring atoms or bicyclic ring of 8 to 10 ring atoms, the cyclic or bicyclic ring having 1, 2, or 3 ring atoms independently chosen from N, O, and S, wherein the cyclic or bicyclic ring is substituted with 0-2 substituents independently chosen from halogen, oxo, cyano, —C₁-C₆alkyl, —C₁-C₆alkoxy, —C₁-C₆haloalkyl, and —C₁-C₆haloalkoxy;
R¹⁶ and R¹⁷ are each independently hydrogen, —C₁-C₆alkyl, —C₂-C₆alkenyl, —C₂-C₆alkynyl, —C₀-C₆alkyl-C₁-C₆alkoxy, —C₀-C₆alkylCOR¹⁸, —C₀-C₆alkyl-COOR¹⁸, —C₀-C₆alkyl-C₂-C₆alkenyl, C₀-C₆alkyl-C₂-C₆alkynyl, —C₀-C₆alkyl(aryl), or —C₀-C₆alkyl(heteroaryl), wherein groups except hydrogen are optionally substituted with halogen, cyano, nitro, oxo, —C₀-C₆alkylCOOR¹⁸, —C₁-C₆alkyl, —C₁-C₆haloalkyl, —C₃-C₇cycloalkyl, a heterocycloalkyl, a heteroaryl, or an aryl;
or R¹⁶ and R¹⁷ along with the N form a cyclic or bicyclic heterocyclic ring of 8 to 10 ring atoms, having 1, 2, or 3 ring atoms independently chosen from N, O, and S, wherein the cyclic or bicyclic heterocyclic ring is substituted with 0-2 substituents independently chosen from halogen, cyano, oxo, —C₁-C₆alkyl, —C₁-C₆alkoxy, —C₁-C₆haloalkyl, and —C₁-C₆haloalkoxy;
R¹⁵ and R¹⁸ are each independently is hydrogen, —C₁-C₆alkyl, —C₁-C₆haloalkyl, —C₀-C₆alkyl(C₃-C₇cycloalkyl), —C₁-C₆alkyl-C₁-C₆alkoxy, —C₁-C₆alkyl-C₂-C₆alkenyl, or —C₁-C₆alkyl-C₂-C₆alkynyl; and
q and p are each independently an integer of 1, 2, 3, 4, or 5.

In another aspect, the compound of Formula II is a compound of Formula IIb,

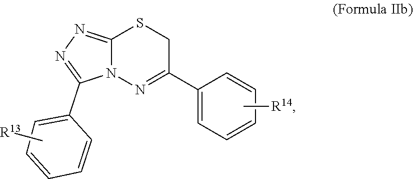

or a pharmaceutically acceptable salt thereof, wherein R¹³ and R¹⁴ are as previously defined.

In another aspect, the compound of Formula II is a compound of Formula IIc,

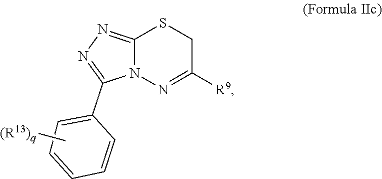

or a pharmaceutically acceptable salt thereof, wherein R¹³, R⁹, and q are as previously defined.

In another aspect, the compound of Formula IIc is a compound of Formula IIIc-1,

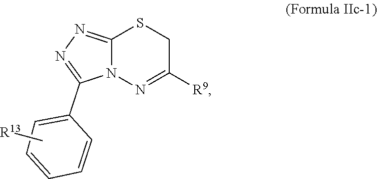

or a pharmaceutically acceptable salt thereof wherein R¹³ and R⁹ are as previously defined.

The compounds of Formula II may be prepared by condensation of suitably substituted 4-amino-(2-alkoxyphenyl)-4H-1,2,4-triazole-3-thiols and alpha-chloro or bromoacetophenones.

In another aspect, described is a method of treating a patient in need of treatment with a CXCR4 agonist, the method comprising administering to the patient an effective amount of the compound of Formula III,

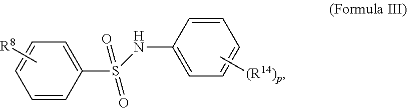

or a pharmaceutically acceptable salt thereof, wherein in Formula III R⁸, R¹⁴, and p are as previously defined. The method of treating a patient in need of treatment with a CXCR4 agonist includes treating a wound and promoting healing of a wound, specifically treating a diabetic wound and promoting the healing of a diabetic wound.

In another aspect, the compound of Formula III is a compound of Formula IIIa,

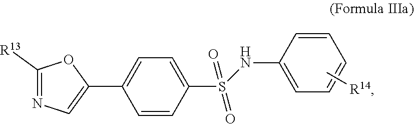

or a pharmaceutically acceptable salt thereof, wherein in Formula IIIa R¹³ and R¹⁴ are as previously defined.

The compounds of Formula I, Formula II, Formula III, a subformula thereof, or a salt thereof, as well as pharmaceutical compositions comprising the compounds, are useful for treating a patient with a wound, specifically a diabetic wound. The method of promoting wound healing including diabetic wound healing comprises providing to a patient in need thereof an effective amount of a compound of Formula I, Formula II, Formula III, a subformula thereof, or a salt thereof. In an embodiment the patient is a mammal, and more specifically a human. The disclosure also provides methods of treating non-human patients such as companion animals, e.g. cats, dogs, and livestock animals. An effective amount of the compound may be an amount sufficient to inhibit the progression of a wound, specifically a diabetic wound; or cause a regression of a wound, specifically a diabetic wound.

An effective amount of a compound or pharmaceutical composition described herein will also provide a sufficient concentration of a compound of Formula I, Formula II, Formula III, a subformula thereof, or a pharmaceutically acceptable salt thereof when administered to a patient. A sufficient concentration is a concentration of the compound in the patient's body suitable for promoting wound healing. Such an amount may be ascertained experimentally, for example by assaying blood concentration of the compound, or theoretically, by calculating bioavailability.

Methods of treatment include providing certain dosage amounts of a compound of Formula I, Formula II, Formula III, a subformula thereof, or a pharmaceutically acceptable salt thereof to a patient. Dosage levels of each compound of from about 0.1 mg to about 140 mg per kilogram of body weight per day are useful in the treatment of (diabetic) wound healing (about 0.5 mg to about 7 g per patient per day). The amount of compound that may be combined with the carrier materials to produce a single dosage form will vary depending upon the patient treated and the particular mode of administration. Dosage unit forms will generally contain between from about 1 mg to about 500 mg of each active compound. In certain embodiments 25 mg to 500 mg, or 25 mg to 200 mg of a compound of Formula I, Formula II, Formula III, a subformula thereof, or a pharmaceutically acceptable salt thereof are provided daily to a patient. Frequency of dosage may also vary depending on the compound used and the nature of the wound treated. However, for treatment of most diabetic wounds, a dosage regimen of 4 times daily or less can be used and in certain embodiments a dosage regimen of 1 or 2 times daily is used.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the wound undergoing treatment.

A compound of Formula I, Formula II, Formula III, a subformula thereof or a pharmaceutically acceptable salt thereof may be administered singularly (i.e., sole therapeutic agent of a regime) to promote wound healing and treat diabetic wounds or may be administered in combination with another active agent, including another compound described herein. One or more compounds of Formula I, Formula II, Formula III, a subformula thereof or a pharmaceutically acceptable salt thereof may be administered in coordination with a regime of one or more other therapeutic agents depending on the health of a patient with the wound.

The CXCR4 agonist compounds described herein may be administered orally, topically, parenterally, by inhalation or spray, sublingually, transdermally, via buccal administration, rectally, as an ophthalmic solution, or by other means, in dosage unit formulations containing conventional pharmaceutically acceptable carriers. The pharmaceutical composition may be formulated as any pharmaceutically useful form, e.g., as an aerosol, a cream, a gel, an ointment, a pill, a capsule, a tablet, a syrup, a transdermal patch, or an ophthalmic solution. Some dosage forms, such as tablets and capsules, are subdivided into suitably sized unit doses containing appropriate quantities of the active components, e.g., an effective amount to achieve the desired purpose.

In an embodiment, the CXCR4 agonist compound is administered topically, optionally in the form of a topical formulation.

In an embodiment, the CXCR4 agonist compound is administered locally in the form of an injectable, specifically intradermally.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, for example, buffering agents, coloring agents, diluents, disintegrants, emulsifiers, flavorants, glidants, lubricants, preservatives, stabilizers, surfactants, tableting agents, and wetting agents. Some carriers may be listed in more than one class, for example vegetable oil may be used as a lubricant in some formulations and a diluent in others.

Methods of treatment provided herein are also useful for treatment of mammals other than humans, including for veterinary applications such as to treat horses and livestock e.g. cattle, sheep, cows, goats, swine and the like, and pets (companion animals) such as dogs and cats.

In an embodiment, a method of treating a diabetic wound in a patient identified as in need of such treatment, the method comprising providing to the patient an effective amount of a compound of Formula I, Formula II, Formula III, a subformula thereof, or a pharmaceutically acceptable salt thereof. The compounds and salts of Formula I, Formula II, Formula III, or subformula thereof provided herein may be administered alone, or in combination with one or more other active agent. In one or more embodiments, the patient is a diabetic patient. In some other embodiments, the patient is with a diabetic wound. Yet, in some other embodiments, the patient is with a decreased expression of stromal cell-derived factor-1α (SDF-1α) relative to non-diabetic patients or wounds.

In one or more embodiments, the compound of Formula I, Formula II, Formula III, a subformula thereof or a pharmaceutically acceptable salt thereof is a CXC chemokine receptor type 4 (CXCR4) agonist.

In an embodiment, a method of promoting wound healing in a patient identified as in need of such treatment comprises providing to the patient an effective amount of a compound of Formula I, Formula II, Formula III, a subformula thereof, or a pharmaceutically acceptable salt thereof. In one aspect, the promotion of wound healing is monitored by measuring the levels of miR-15b expression or miR-29a expression or a ratio of wound size to a day zero wound size in the subject. Typically levels of miR-15b or miR-29a are measured prior to treatment, wherein an elevated level is indicative of the need to use a CXCR4 agonist compound to treat the diabetic wound. Once the elevated levels are established, the level of miR-15b or miR-29a is determined during the course of and/or following termination of treatment to establish efficacy. In certain embodiments, the level of miR-15b or miR-29a is only determined during the course of and/or following termination of treatment A reduction of miR-15b or miR-29a levels during the course of treatment and following treatment is indicative of efficacy. Similarly, a determination that miR-15b or miR-29a levels are not elevated during the course of or following treatment is also indicative of efficacy. Typically, these miR-15b or miR-29a measurements will be utilized together with other well-known determinations of efficacy of wound healing treatment, such as reduction in number and size of wounds and/or other diabetes-associated lesions, improvement in the general health of the subject, and alterations in other biomarkers that are associated with diabetic wound healing treatment efficacy.

### EXAMPLES

### Example 1

### Application of Cell-Based High-Throughput Screening

In an effort to identify CXCR4 activators from the NIH Molecular Library Small Molecule Repository (MLSMR), a primary screening assay was developed to measure β-arrestin recruitment to the activated G-protein coupled receptor by Florescence Resonance Energy Transfer (FRET) in U2OS cells overexpressing the CXCR4 receptor engineered with a β-lactamase (bla) reporter system (Invitrogen cat #K1779). CXCR4-bla cells plated in 1536 w were stimulated in the presence of 10 uM compound concentration. The quantity of arrestin binding was detected by decrease in FRET-enabled β-lactamase substrate (460 nm/535 nm) and normalized to maximal amount produced by an EC₈₀ concentration of SDF-1α. Using this approach, 370,620 compounds from the NIH MLSMR were successfully screened and 303 hits were identified (hit rate=0.08%; Z′=0.83). Hit confirmation resulted in 130 compounds as potential CXCR4 activators (response >40% activity and filtered for florescence interference in the 535 nm channel). These compounds were graded according to activity and structure resulting in 86 compounds that met criteria as potential CXCR4 agonists for further characterization.

### Identified_Leadmcompounds by Structure-Activity Studies

Commercially available compounds representing three distinct scaffolds were purchased and EC₅₀ values were determined in the primary 1536 w β-arrestin recruitment assay using SDF-1α from R&D systems. Chemical stability, synthetic tractability, solubility and complete dose response and potency criteria led to the prioritization of the 5-aryl-2-cyclopropyl oxazole scaffold, represented by primary hit Compound 2 (Table 1). Early structure-activity relationship was established around the 5-aryl-2-cyclopropyl oxazole core with commercially available analogs with diverse substituents in the sulfonanilide ring of the scaffold. Electron-rich alkoxy substituents at 2-position were most promising in terms of activity among monosubstituted sulfonanilide derivatives and 2-ethoxy analog had an EC₅₀ of 1.0 μM (Table 1 entries 5 vs 1-7). Moving the alkoxy substituent to 3- and 4-position led to loss of activity, indicating that the 2-alkoxy substituents might lock the anilide ring in a favored conformation (entries 8-9). Screening of several dimethyl substituents (entries 10-15) confirmed the above observation and 2,5-dimethyl, 13 and 2,6-dimethyl analog, 14 were found to be the most active among the dimethyl analogs due to their potential ability to force the anilide ring in a favorable non-planar orientation compared to the rest of the molecule. Keeping the 2-OMe substituent constant a small set of electron withdrawing and electron rich substituents at the 5-position of the anilide ring were screened (entries 16-19). Electron rich groups appear to be better suited at the 5-position and 2,5-dimethoxy analog, 19 (UCUF-728) had the best activity and agonist response in the 1536 w screening platform (EC₅₀=1.1 μM and Eₘₐₓ=60%). The 3,4 and 3,5-dimethoxy analogs were inactive or had marginal activity further confirming the observations (entries 20 and 21).

TABLE 1
SAR of analogs of the 5-aryl-2-cyclopropyloxazole series in the
primary CXCR4 b-arrestin recruitment assay.

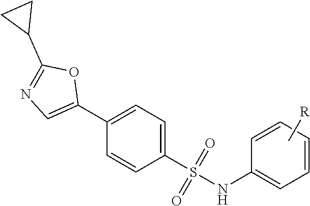

Table 1: SAR of 5-aryl-2-cyclopropyloxazole series

| | | CXCR4 b- | CXCR4 |
|---|---|---|---|
| | | arrestin | b-arrestin |
| Oxazole Entry | R | EC₅₀ (mM) | Eₘₐₓ (%) |
| 1 | H | >80 | |
| 2 | 2-Me | 5.9 | 27 |
| 3 | 2-Et | 2.6 | 38 |
| 4 | 2-OMe | 6.1 | 45 |
| 5 | 2-OEt | 1.0 | 46 |
| 6 | 2-Cl | 22 | 35 |
| 7 | 2-F | >80 | |
| 8 | 3-OMe | >80 | |
| 9 | 4-OEt | 44.3 | 38 |
| 10 | 2,3-dimethyl | 1.9 | 40 |
| 11 | 3,4-dimethyl | >10 | |
| 12 | 2,4-dimethyl | 3.4 | 38 |
| 13 | 2,5-dimethyl | 0.7 | 42 |
| 14 | 2,6-dimethyl | 0.6 | 45 |
| 15 | 3,5-dimethyl | 3.1 | 39 |
| 16 | 2-OMe, 5-Cl | 1.65 | 47 |
| 17 | 2-OMe, 5-F | 7.3 | 42 |
| 18 | 2-OMe, 5-Me | 0.9 | 48 |
| 19 (UCUF-728) | 2,5-dimethoxy | 1.1 | 60 |
| 20 | 3,4-dimethoxy | >80 | |
| 21 | 3,5-dimethoxy | 5.6 | 48 |

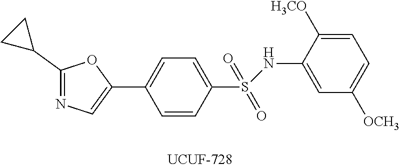 [SMILES: COc1ccc(OC)c(NS(=O)(=O)c2ccc(-c3cnc(C4CC4)o3)cc2)c1]

4-(2-Cyclopropyloxazol-5-yl)-N-(2,5-dimethoxyphenyl)benzenesulfonamide (Oxazole 19, UCUF-728): ¹H NMR Spectra of UCUF-728. 1H NMR (600 MHz, CDCl3) δ 7.80 (d, J=8.6 Hz, 2H), 7.60 (d, J=8.6 Hz, 2H), 7.19 (d, J=3.0 Hz, 1H), 7.08 (s, 1H), 6.67 (d, J=8.9 Hz, 1H), 6.58 (dd, J=8.9, 3.0 Hz, 1H), 3.77 (s, 3H), 3.62 (s, 3H), 2.18-2.06 (m, 1H), 1.19-1.06 (m, 4H). Mass Spectrometry of UCUF-728. Chemical Formula: C₂₀H₂₀N₂O₅S Calculated [M+H]: 401.12.

### In Vitro Biological Profile of UCUF-728

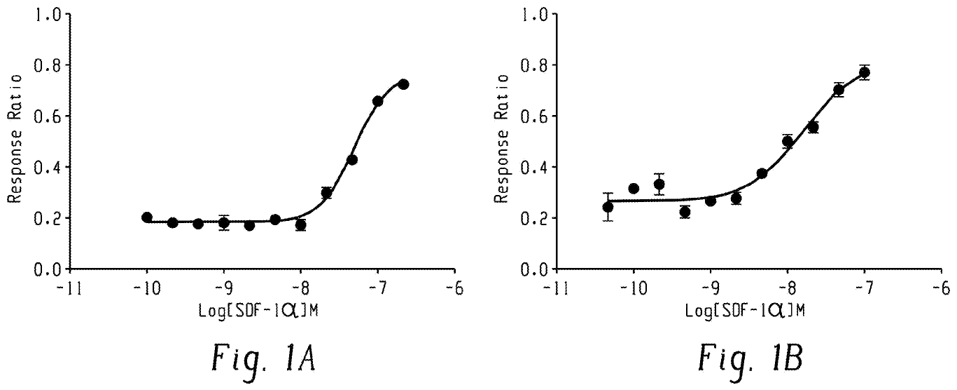

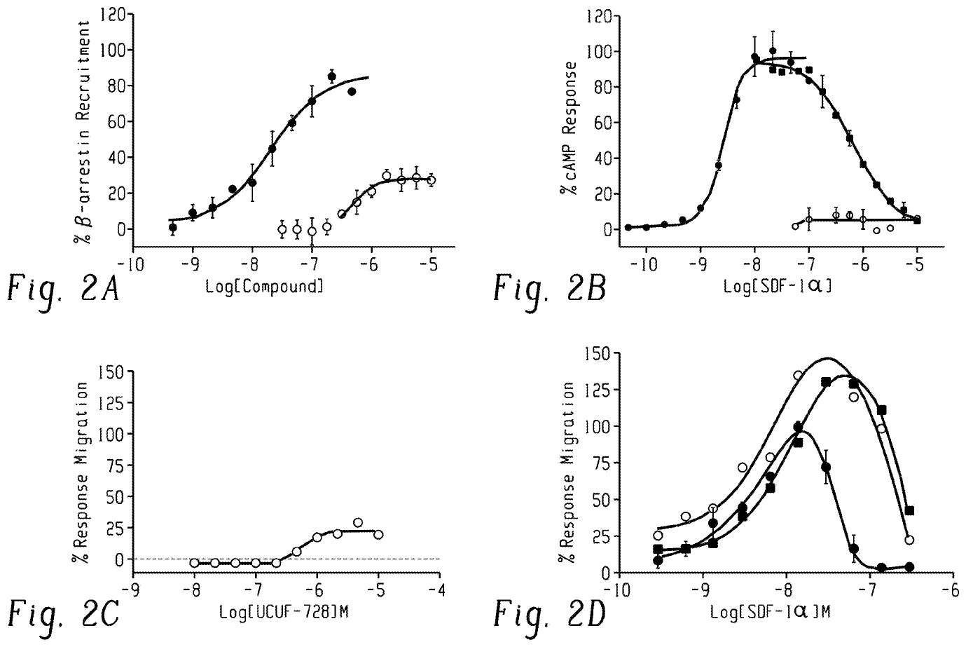

UCUF-728 was further characterized in 384 w assays using SDF-1α from a different source than was used in the high throughput screening assay. The new ligand was 3-fold more potent in the primary assay and was more economical (FIGS. 1A and 1B). Using this ligand source, UCUF-728 confirmed activation of β-arrestin recruitment with EC₅₀=0.5 μM and Eₘₐₓ=30% normalized to SDF-1α (FIG. 2A).

The CXCR4 receptor is a G-protein coupled receptor (GPCR) coupled to the Gi class, whose primary role is inhibition of adenylate cyclase. The ability of UCUF-728 to induce Gi-mediated cAMP-inhibitory cellular response was tested. SDF-1α inhibited forskolin-induced adenylate cyclase in CHO-K1 CXCR4 overexpressed cells and this response was dose-dependently blocked by AMD3100, a small bicyclam molecule that inhibits the binding of SDF-1α to CXCR4 (FIG. 2B). However, UCUF-728 did not inhibit forskolin-induced adenylate cyclase in the overexpressed cell line indicating biased signaling for b-arrestin recruitment over cAMP signaling (FIG. 2A and FIG. 2B).

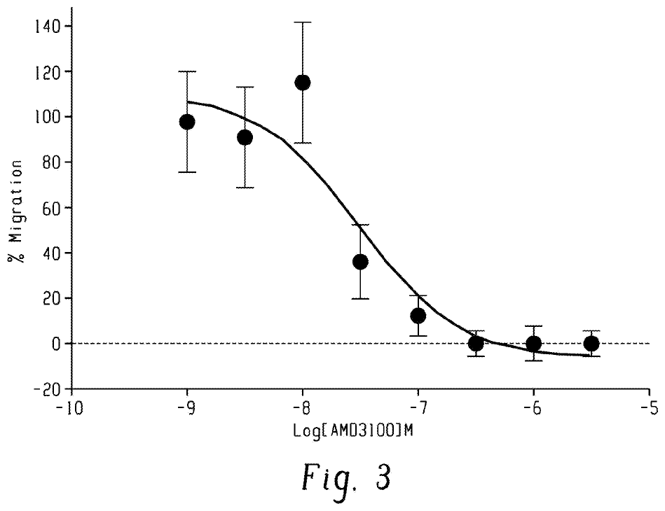

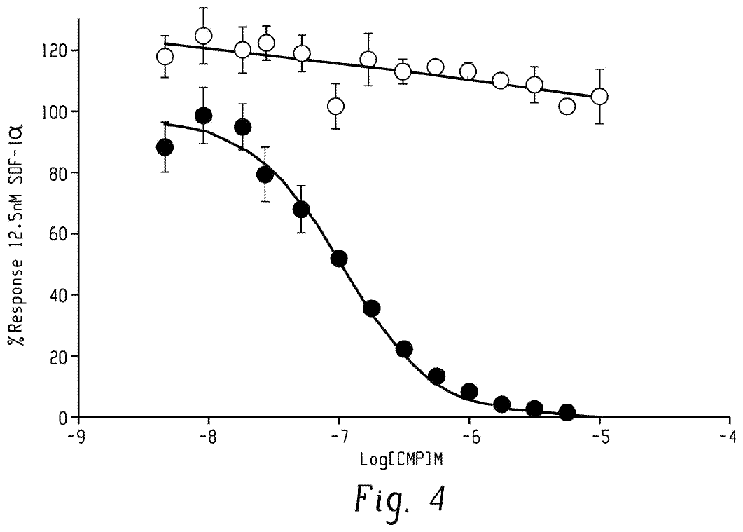

SDF-1α stimulates chemotaxis in a high percentage of resting and active T lymphocytes and CXCR4 receptor is highly expressed in the CEM (Leukemia) cell line. Thus, to confirm CXCR4 functional activity, UCUF-728 was evaluated in a 96 w transwell migration assay in CEM human lymphoblast cells. Addition, of lymphoblasts to the upper chamber and UCUF-728 to the lower channel induced migration with similar EC₅₀ and Eₘₐₓ as the B-arrestin recruitment activity (FIG. 2A and FIG. 2C). The chemoattractant SDF-1α in the lower channel induced migration in the typical bell-shaped curve (FIG. 2D) and this activity was blocked by addition of AMD3100 (FIG. 3). Interestingly, the SDF-1α-mediated response was potentiated in the presence of saturating concentrations of UCUF-728 (FIG. 2D) suggesting that UCUF-728 is a potential positive allosteric activator. A partial agonist is expected to inhibit activity of a full agonist if both agonists bind to the same site on the receptor, the results herein suggested that UCUF-728 may not orthosterically bind CXCR4 receptor. To confirm this, UCUF-728 was tested in a CXCR4 Tag-lite binding assay and UCUF-728 did not displace SDF-1α binding, whereas, AMD3100 dose-dependently displaced SDF-1α with potency comparable to literature values (FIG. 4). From the analog structure-activity relationship studies and characterization studies in the chemotaxis assay, cAMP signaling and CXCR4 binding assay, UCUF-728 was identified as a potent and functionally selective CXCR4 receptor modulator.

### Ex Vivo Validation of UCUF-728

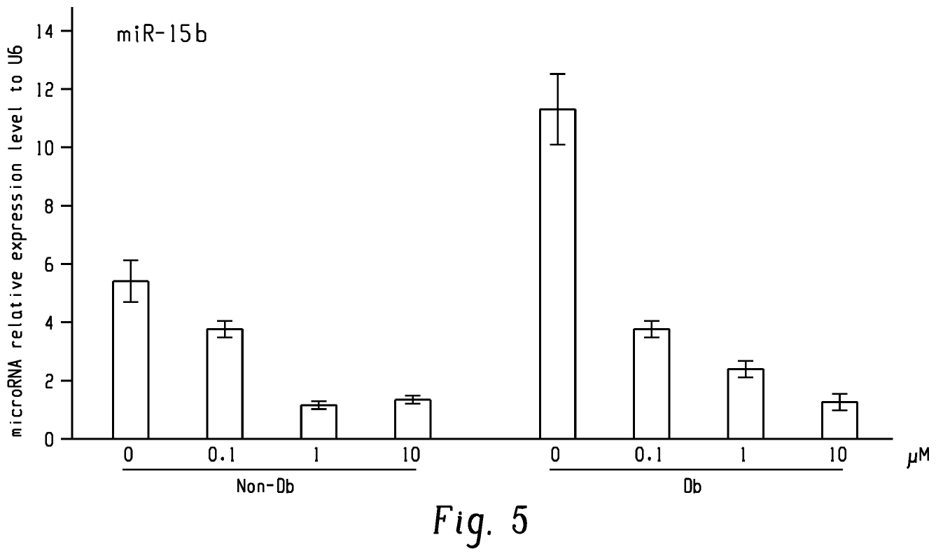

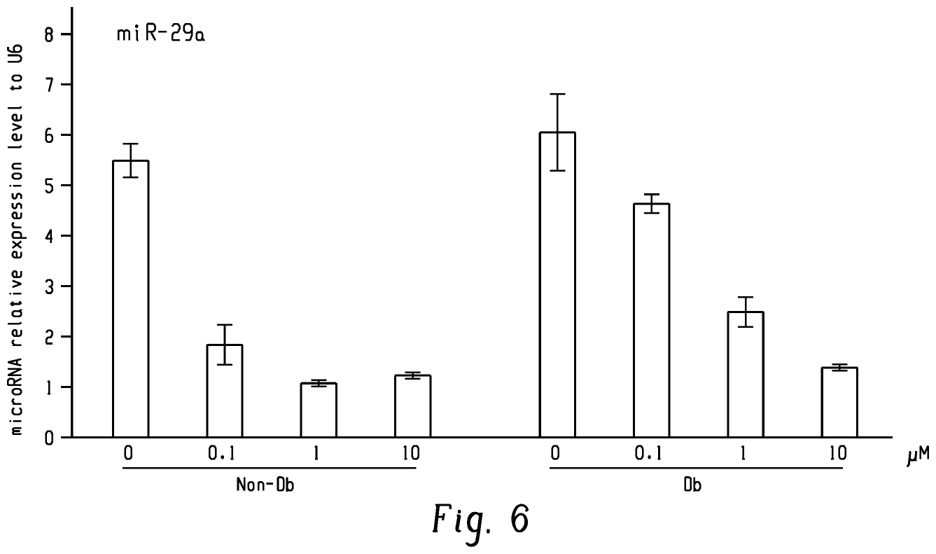

An additional secondary screen was performed to further validate activity of UCUF-728 as a selective CXCR4 receptor modulator. Human diabetic and non-diabetic fibroblasts were cultured with increasing concentrations of the compound. The cells were incubated for 24 hours and then total cellular RNA was isolated to examine the ability of the compound to correct the abnormal expression of miR15b, and miR29a. It has been shown that human diabetic skin has increased miR-15b expression at baseline compared to non-diabetic skin (Xu et al. “The role of microRNA-15b in the impaired angiogenesis in diabetic wounds.” Wound Repair Regen 2014; 22(5):671-7). The effect of UCUF-728 on human diabetic fibroblast expression of miR-15b was examined and it was found that UCUF-728 treatment decreased expression of miR-15b in human diabetic fibroblasts, similar to levels expressed by non-diabetic fibroblasts (FIG. 5). In preliminary studies it has been shown that production of miR-29a is significantly upregulated in human diabetic skin at baseline compared to non-diabetic skin (Zgheib et al. “Mechanisms of mesenchymal stem cell correction of the impaired biomechanical properties of diabetic skin: The role of miR-29a.” Wound Repair Regen 2016; 24(2):237-46). Here, it was found that human diabetic fibroblasts had increased expression of miR-29a compared to non-diabetic fibroblasts, and that UCUF-728 resulted in a significant decrease in miR-29a expression level (FIG. 6).

### In Vivo Application of UCUF-728

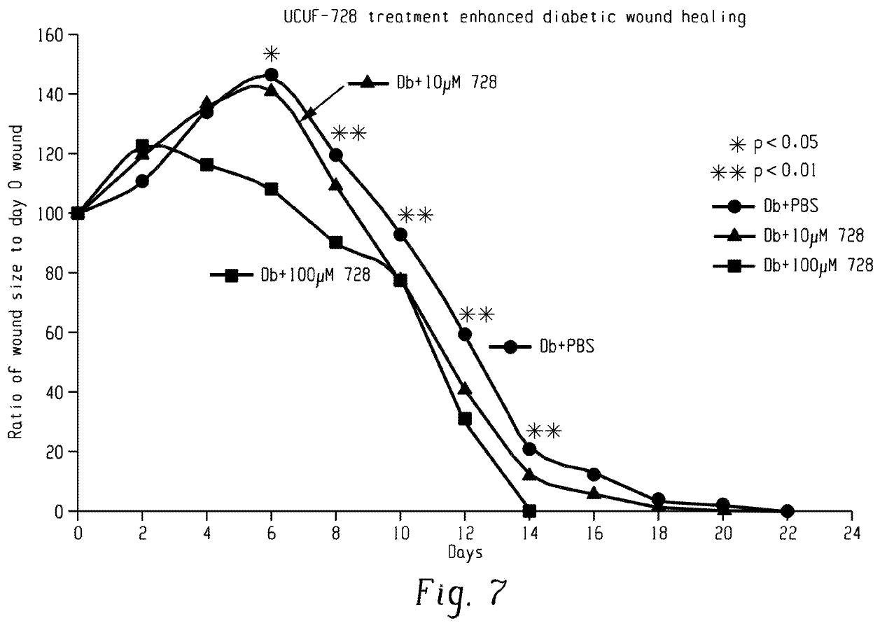

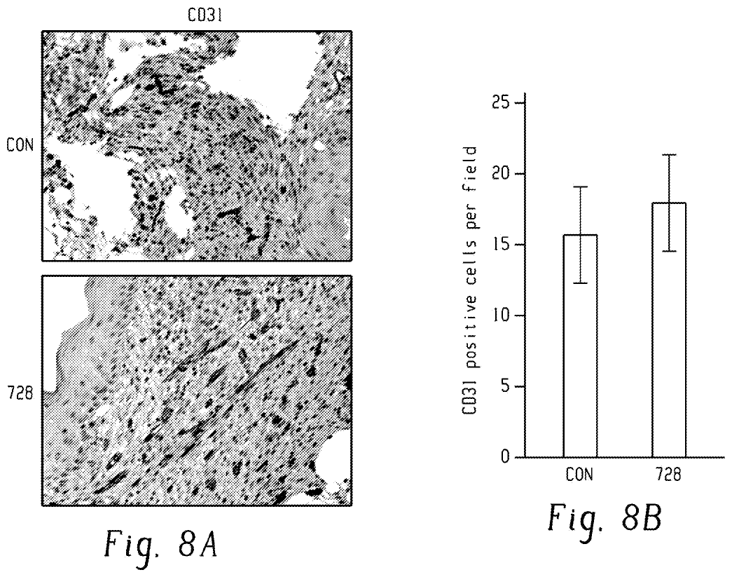

The ability of compound UCUF-728 to improve diabetic wound healing in vivo was examined. Full-thickness excisional 8 mm wounds were created in Db mice and were immediately treated with 10 μM UCUF-728 or phosphate-buffered saline (PBS) control. Wound healing over the course of 22 days was monitored. Initial wound size was calculated immediately after wounding, and wound closure was assessed over time as the percentage of initial wound area. By post-injury day 6, Db wounds treated with UCUF-728 exhibited a decrease in wound surface area compared to Db wounds treated with PBS. The time of full closure was 14 days compared to 22 days in Db wounds treated with PBS, indicating that UCUF-728 treatment in diabetic wounds significantly enhanced diabetic wound healing (FIG. 7). The effect of UCUF-728 treatment on angiogenesis was assessed using immunohistochemistry for endothelial marker CD31. Representative photos of immunoperoxidase staining for CD31 at 7 days in diabetic wounds treated with PBS or UCUF-728 are shown in FIG. 8A. Quantitative analysis of diabetic wounds treated with UCUF-728 demonstrated a significant increase in the number of vessels compared with PBS-treated diabetic wounds (FIG. 8B).

Secretion of the chemokine SDF-1α, with subsequent activation of the CXCR4 receptor, is an important component for effective wound healing. It is a chemokine that promotes recruitment of hematopoietic progenitor cells to areas of tissue injury. Expression of SDF-1α is decreased in diabetic wounds, which may underlie the wound healing impairment depicted by an increased wound closure time, decreased granulation tissue, and a larger epithelial gap. Reduced cellular migration resulting from decreased SDF-1a expression may contribute to observed healing impairment, as non-diabetic mouse wounds lacking lymphocytes recapitulated features of impaired wound healing, including preferential M1 polarization, increased basal ROS levels, and reduced angiogenesis. Theses mechanistic insights highlight the utility of exploring novel therapeutics that can circumvent deficits in CXCR4 receptor activation to correct healing impairment.

Herein is a high-throughput β-arrestin recruitment assay to screen compounds for potential utility as CXCR4 receptor activators. Subsequent structure-activity relationship (SAR) studies identified a chemical scaffold that functions as a CXCR4 agonists (UCUF-728) and was further confirmed activity with in vitro and in vivo validation studies. Treatment of human diabetic fibroblasts with UCUF-728 resulted in potent suppression of miR-15b expression, an outcome observed with the lowest concentration (0.1 μM). MiR-15b is a negative modulator of angiogenesis that is upregulated in diabetic wounds during the early phase of healing. Increased expression of miR-15b is associated with decreased expression of proangiogenic target genes including vascular endothelial growth factor (VEGFα), hypoxia inducible factor (HIF-1α), and B-cell lymphoma 2 (BCL2). As previously demonstrated, therapeutic suppression of miR-15b expression in diabetic wounds may contribute to accelerated wound closure by enhancing angiogenesis. UCUF-728 treatment also reduced miR-29a expression in diabetic fibroblasts, but relative suppression was less potent than that observed of miR-15b. MiR-29a is upregulated in human and murine diabetic skin. Evidence suggests that dysregulation of miR-29a contributes to decreased collagen I protein content in diabetic wounds, leading to impaired biomechanical properties of skin that may underlie increased susceptibility to injury.

In vivo, UCUF-728 treatment reduced wound closure time by 36% which was observed alongside enhanced angiogenesis in treated diabetic wounds. Together, these studies suggest that activation of CXCR4 receptors with UCUF-728 accelerates wound healing by favoring promotion of angiogenesis via suppression of miR-15b. Mild reductions in miR-29a were observed, with near normalization of miR-29a expression to nondiabetic control levels (no treatment). This suggests that promotion of collagen I protein content via suppression miR-29a, could also be contributing to UCUF-728-mediated wound repair. Together, this work demonstrates the clinical potential of small molecule CXCR4 agonists as novel therapies for wound healing in diabetes.

The development of topical therapies for treating impaired wound healing and reducing the probability of reinjury is envisioned.

### Thiadiazine Compounds

### Synthesis of 3-(2-ethoxyphenyl)-6-(4-(pyrrolidin-1-yl)phenyl)-7H-[1,2,4]triazolo[3,4-b][1,3,4]thiadiazine (UCUF-965; Table 2 Entry 23/24)

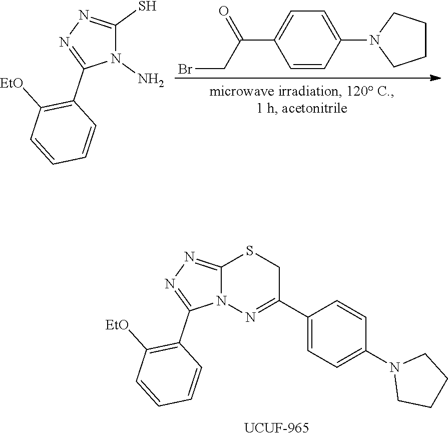 [SMILES: CCOc1ccccc1-c1nnc(S)n1N ; O=C(CBr)c1ccc(N2CCCC2)cc1 ; CCOc1ccccc1-c1nnc2n1N=C(c1ccc(N3CCCC3)cc1)CS2]

A microwave reaction tube was charged with 4-amino-5-(2-ethoxyphenyl)-4H-1,2,4-triazole-3-thiol (0.25 g, 1.06 mmol) and 2-bromo-1-(4-(pyrrolidin-1-yl)phenyl)ethan-1-one (0.284 g, 1.06 mmol). To the mixture, 3 mL acetonitrile and the tube was sealed. The reaction was heated at 120° C. for 1 h. A yellow brown precipitate was observed. The seal was opened and reaction mixture was cooled to room temperature. 2-3 mL ethyl acetate was added to precipitate more product. The product was filtered and washed extensively with ethyl acetate (5 mL, 5×) and air-dried to yield 3-(2-ethoxyphenyl)-6-(4-(pyrrolidin-1-yl)phenyl)-7H-[1,2,4]triazolo[3,4-b][1,3,4]thiadiazine (UCUF-965) as a bright yellow solid (0.362 g, 84%). ¹H NMR (600 MHz, DMSO) δ 7.76-7.67 (m, 2H), 7.57-7.47 (m, 2H), 7.17 (d, J=8.3 Hz, 1H), 7.09 (td, J=7.4, 0.9 Hz, 1H), 6.61-6.56 (m, 2H), 4.27 (s, 2H), 3.98 (q, J=6.9 Hz, 2H), 3.31-3.26 (m, 4H), 3.16 (s, 1H), 1.99-1.91 (m, 4H), 1.03 (t, J=6.9 Hz, 3H). ¹³C NMR (151 MHz, DMSO) δ 156.83, 155.38, 150.02, 142.61, 132.67, 131.26, 129.02, 120.33, 118.32, 114.28, 112.61, 111.60, 63.73, 48.59, 47.25, 24.92, 22.56, 14.29. LRMS, ESI(+ve) calculated for C₂₂H₂₃N₅OS, [M+H]=406.17, observed [M+H]=406.15.

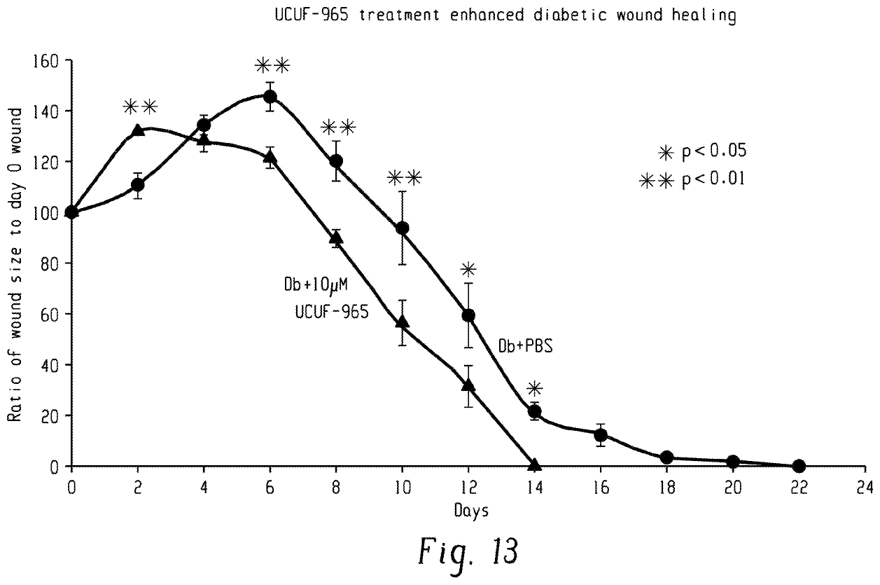

Diabetic and non-diabetic fibroblasts were cultured with increasing concentrations of a UCUF-965 compound. The cells were incubated for 24 hours and then total cellular RNA was isolated to examine the ability of the compound to correct the abnormal expression of miR15b, and miR29a. Angiogenesis is regulated by hypoxia and the production of growth factors such as Hypoxia-inducible Factor 1-alpha (HIF-1a) and Vascular Endothelial Growth Factor (VEGF). Diabetic wounds have been shown to have decreased production of both HIF-1α and VEGF. MicroRNA-15b (miR-15b) has been described as a key negative modulator of angiogenesis. Under hypoxia, miR-15b is repressed, resulting in an increased level of VEGF. Over-expression of miR-15b also markedly attenuates hypoxia induced vessel tube formation and cell migration. MiR-15b in diabetic wounds is significantly upregulated and is associated with decreased VEGF production and vessel formation, compared to non-diabetic wounds. In preliminary studies it has been shown that human diabetic skin has increased miR-15b expression at baseline compared to non-diabetic skin (FIG. 13). The effect of UCUF-965 on human diabetic fibroblast expression of miR-15b was studied and it was found that UCUF-965 treatment decreased expression of miR-15b in human diabetic fibroblasts, similar to levels expressed by nondiabetic fibroblasts (FIG. 13). Production of extracellular matrix (ECM) is a key component of wound healing as well as maintenance of tissue homeostasis and skin integrity. The predominant ECM molecule responsible for wound healing and skin integrity is Type I collagen. Diabetic wounds have been shown to have impaired production of type I collagen, and it has been shown that diabetic skin also has decreased collagen I protein at baseline. MicroRNA-29a (miR-29a) negatively regulates type I collagen protein production by suppressing translation of col1A1 mRNA. In preliminary studies it has been shown that production of miR-29a is significantly upregulated in human diabetic skin at baseline compared to nondiabetic skin (FIG. 13). In additional preliminary studies, it has been found that human diabetic fibroblasts produced significantly more miR-29a than non-diabetic fibroblasts, and that UCUF-965 resulted in a significant decrease in miR-29a (FIG. 13).

The ability of UCUF-965, a thiadiazine compound, to improve diabetic wound healing in vivo was examined. Full-thickness excisional 8 mm wounds were created in Db mice and were immediately treated with 10 μM UCUF-965 or PBS control. Wound healing over the course of 22 days was monitored. Initial wound size was calculated immediately after wounding, and wound closure was assessed over time as the percentage of initial wound area. FIG. 13 indicated the wound healing effect of UCUF-965 topical administration on excision wound model on different days after injury. By day 6 after wounding, Db wounds treated with UCUF-965 exhibited a decrease in wound surface area compared to Db wounds treated with PBS (**p<0.01), indicating that UCUF-965 treatment in diabetic wounds enhanced wound healing by 36% at 10 μM over PBS control (FIG. 13). UCUF-965-treated diabetic wounds closed around 14 days after treatment whereas control PBS-treated wounds healed at day 22. UCUF-965 is a promising positive allosteric modulator of CXCR4 signaling to treat diabetic would healing.

UCUF-965 is a potent allosteric activator of β-arrestin recruitment and inhibitor of cAMP production in CXCR4 receptor overexpressing cell lines. UCUF-965 potentiates the CXCL12 agonist response in cAMP signaling pathway, activates CXCL12 stimulated migration in lymphoblast cells, exhibits agonist activity in calcium signaling, and modulates the levels of specific microRNA involved in the complex wound repair process, specifically in mouse fibroblasts. Furthermore, UCUF-965 enhanced angiogenesis markers and reduced wound healing time by 36% at 10.0 μM in diabetic mice models compared to untreated control.

### Reagents and Drug Treatment Protocol.

All compounds were maintained as 10 mM DMSO stocks. To determine effects of compounds screened in cell-based assays, selected compounds were added to respective plates using the TECAN D300e digital dispenser. DMSO concentration was constant across all assay wells and did not exceed 0.5% in any cell-based assay. Unless otherwise indicated, reagents were purchased from Thermo Fisher Scientific (Waltham, MA).

### Cell Culture—Oxazole Scaffold.

CEM-CCRF cells (ATCC CCL-119) were cultured in growth media consisting of RPMI 1640 (Coming; Coming NY) supplemented with 10% Fetal Bovine Serum (Coming; Coming NY), and 100 IU penicillin, 100 mg/ml streptomycin sulfate (Corning; Corning NY). Cells were maintained in suspension and subcultured twice weekly to maintain cell densities between 0.8×106 cells/mL and 2.5×10⁶ cells/mL to maintain exponential growth phase. Cells were harvested for use in migration and calcium flux assays at 2.0×10⁶ cells/mL to 2.5×10⁶ cells/mL and resuspended in assay media to desired density for respective experiments. Tango™ CXCR4-bla U2OS Cells were maintained as a monolayer culture in McCoy's 5a (Corning; Corning NY) supplemented with 10% dialyzed Fetal Bovine Serum, 0.1 mM MEM Non-essential amino acids solution, 25 mM HEPES solution (Corning; Corning NY), 1 mM Sodium Pyruvate, 200 ug/mL Zeocin selection reagent, 50 ug/mL Hygromycin B, and 100 ug/mL Geneticin. U2OS cells were routinely subcultured twice weekly by 0.25% Trypsin-EDTA treatment (Corning; Corning, NY) and passaged to continue exponential growth phase for cell signaling assays. All cell cultures were maintained in cell culture incubators at 37° C. with 5% CO2. Human Dermal Fibroblasts were isolated from human skin biopsies according to the method Kisiel MA, Klar AS. Isolation and Culture of Human Dermal Fibroblasts. Methods Mol Biol 2019; 1993:71-8). Human dermal fibroblasts were cultured in full medium comprising Dulbecco's modified eagle high-glucose (DMEM, Sigma-Aldrich, St. Louis, MO) supplemented with 10% fetal bovine serum (FBS) and maintained at 37° C. in a humidified atmosphere containing 5% CO₂. For further experiments, cells were seeded cultured for 12 hr. Thereafter, cells were starved for 16 hr and were stimulated with UCUF-728 at different dose.

Cell Culture—thiadiazine scaffold CXCR4 agonists.

Tango™ CXCR4-bla U2OS Cells (Invitrogen; K1779) were used for (β-arrestin recruitment assays and maintained as a monolayer culture in McCoy's 5a (Corning; 10-050-CV) supplemented with 10% dialyzed Fetal Bovine Serum (Gibco; 26400-044), 0.1 mM MEM Non-essential amino acids solution, 25 mM HEPES solution (Corning; 25-060-CI), 1 mM Sodium Pyruvate (Gibco; 11360-070), 200 μg/mL Zeocin selection reagent (Gibco; R25001), 50 μg/mL Hygromycin B (Gibco, 10687010), and 100 μg/mL Geneticin (G418 Sulfate) (Gibco, 10131035). U2OS cells were subcultured twice weekly by 0.05% Trypsin-EDTA treatment (Corning; 25-051-CI) and passaged to continue exponential growth phase for cell signaling assays. All cell cultures were maintained in cell culture incubators at 37° C. with 5% CO₂. CHO-K1 CXCR4 cells (DiscoverX, Fremont, CA, USA) were used for cAMP inhibition assays and maintained as a monolayer culture in DMEM/F12 (Corning; 10-091-CV) supplemented with 10% Fetal Bovine Serum (Corning; 35-011-CV) and 100IU penicillin, 100 mg/mL streptomycin sulfate (Corning; 30-002-CI) and 50 mg/mL G418 (Gibco; 101310350. Cells were subculture 2 times weekly by o.o5% Trypsin-EDTA treatment (Corning; 25-051-CI) and passaged to continue exponential growth phase for cell signaling assays. All cell cultures were maintained in cell culture incubators at 37° C. with 5% CO2.CEM-CCRF cells (ATCC CCL-119) were used in both the migration and calcium flux assays and were cultured in growth media consisting of RPMI 1640 (Corning; 10-040-CV) supplemented with 10% Fetal Bovine Serum (Corning; 35-011-CV), and 100 IU penicillin, 100 mg/ml streptomycin sulfate (Corning; 30-002-CI). Cells were maintained in suspension and sub-cultured twice weekly to maintain cell densities between 0.8×10⁶ cells/mL and 2.5×10⁶ cells/mL to maintain exponential growth phase. Cells were harvested for use in migration and calcium flux assays at 2.0×10⁶ cells/mL to 2.5×10⁶ cells/mL and resuspended in assay media to desired density for respective experiments.

### B-Arrestin Recruitment.

Compounds, SDF-1α (PeproTech; 30028A), and DMSO (Sigma Aldrich; St. Louis, MO) normalization to 0.5% were dispensed using the TECAN D300e digital dispenser into a 384-well black wall clear-bottom microplate (Corning; Corning, NY). Tango™ CXCR4-bla U2OS Cells were resuspended in assay media consisting of DMEM (Corning; Corning, NY) supplemented with 1% dialyzed Fetal bovine serum, 0.1 mM MEM Non-essential amino acids solution, 25 mM HEPES solution (Corning; Corning, NY), 1 mM Sodium Pyruvate and 100 IU penicillin, 100 mg/ml streptomycin sulfate (Corning; Corning, NY) at a density of 3.0×10⁵ cells/ml. Assay media FreeStyle 293 expression Medium (Gibco; 12338018) was used for testing the thiadiazine scaffold CXCR4 agonists. Cells suspension was dispensed at 30 μl per well into assay plate and incubated overnight in a cell culture incubator at 37° C. with 5% CO₂. For testing the thiadiazine scaffold CXCR4 agonists, following overnight incubation, CXCL12 ligand (PeproTech; 30028A), was dispensed manually and cells were incubated for additional 5 hours in a cell culture incubator. Following overnight incubation, LiveBlazer FRET B/G Loading Kit (Invitrogen; K1095) working reagent was prepared according to supplier guidelines and 6 μl of solution was dispensed into each well; 8 μL of solution for testing the thiadiazine scaffold CXCR4 agonists. Assay plate was covered and incubated in a dark place for two hours and Fluorescence Intensity was measured using the BMG LABTECH CLARIOstar Plus (BMG LABTECH Inc., Cary, NC) according to the LiveBlazer FRET B/G Loading Kit excitation and emission measurement guidelines.

cAMP Signaling Assay—Oxazole Scaffold.

AMD3100, UCUF-728 and SDF-1α (PeproTech; Cranbury, NJ) were dispensed in concentration-response mode using the TECAN D300e digital dispenser into white 384-OptiPlates (Perkin Elmer, Waltham, MA). Forskolin at final concentration of 0.5 mM was added to all wells using the TECAN dispenser. 10 ml PathHunter CHO-K1 CXCR4 cells (DiscoverX, Fremont, CA) were added to compounds at 3,000 cells/well, centrifuged at 1000 rpm for 30 sec and incubated for 30 min at room temperature. DMSO (Sigma Aldrich, St. Louis, MO) was normalization to 0.5%. 5 μL of 4× Eu-cAMP tracer working solution was added to all wells followed by addition of 5 μL of 4× Ulight-anti-cAMP working solution to all wells. Plates were centrifuged at 1000 rpm for 1 min and incubated for 1 hr at room temperature then read using CLARIOstar Plus (BMG LABTECH Inc., Cary, NC) Lance CAMP protocol.

### Adenylate Cyclase Assay—Thiadiazine Scaffold CXCR4 Agonists.

Compounds and DMSO normalization to 0.5% were dispensed using the TECAN D300e digital dispenser into the wells of a 384-well white wall clear-bottom assay plate (Griener 781983). CHO-CXCR4-cAMP cells maintained in monolayer culture were prepared in DMEM/F12 (Corning; 10-091-CV) supplemented with 2% Fetal Bovine Serum (Corning; 35-011-CV) at a density of 0.15×10⁶ cells/mL. This cell suspension was dispensed at 20 μL per well into the assay plate and allowed to incubate overnight for approximately 18 hrs with compounds in cell culture incubator at 37° C. with 5% CO₂. Following incubation, DMEM/F12 media was gently removed, then the Lance Ultra cAMP Kit (Perkin Elmer; TRF0263) was performed according to the supplier guidelines. Forskolin was dispensed at 300 nM to elicit cAMP production and CXCR4 natural ligand SDF-1α (CXCL12) (Peprotech;300-28A) was added in titration to determine the maximal cAMP inhibition. Time-resolved fluorescence was measured on the BMG LABTECH CLARIOstar Plus (BMG LABTECH Inc., Cary, NC) using the Lance Ultra cAMP kit guidelines for excitation and emission.

### Migration Assay.

CEM-CCRF (ATCC CCL-119) were suspended in RPMI 1640 (Corning; 10-040-CV) at 5.0×10⁵ cells/mL and allowed to incubate in cell culture incubator at 37° C. with 5% CO₂ for one hour. For agonist activity measurement, compounds, SDF-1α control ligand (PeproTech; 300-28A), and DMSO (Sigma Aldrich, St. Louis, MO) normalization to 0.5% were dispensed into the compound receiver tray of Multi-Screen 96-well assay plate (Millipore Sigma, St. Louis, MO) at desired concentration using the TECAN D300e digital dispenser. Following 1 hr cell incubation and compound dispensing, 150 μl of RPMI 1640 supplemented with 2% Fetal Bovine Serum was added to each well of the compound receiver tray and 50 μl of 5×10⁵ cells/ml cell suspension in RPMI were added to each well of the top filter plate. Plates were reassembled and placed in a cell culture incubator at 37° C. with 5% CO₂ for three hours. After incubation, Multi-Screen plate was disassembled and 100 μl of solution was collected from compound receiver tray and transferred to 96 well luminescent plate (Thermo Scientific; 265302) and an equal volume of ATPLite Istep luminescence reagent (Perkin Elmer, Waltham, MA; 6016731) was added to each well. Luminescence was measured using the CLARIOstar Plus (BMG LABTECH Inc., Cary, NC).

### Calcium Flux Activity.

Fluo-4 Direct Calcium Assay Kit (Invitrogen; F10472) reagents were prepared according to the supplier guidelines at 2× concentration and allowed to equilibrate to room temperature in the dark. CCRF-CEM cells in fresh assay media were used to prepare a new 4.5 mL cell suspension at a density of 4.8×10⁵ cells/mL. An equal volume of 2× Fluo-4 Direct Calcium Assay Kit reagent was added to this cell suspension and placed in a cell culture incubator at for 1 hr. Following incubation, 25 μL of cell suspension loaded with Fluo-4 Direct Calcium reagent was dispensed into a 384-well black wall clear-bottom plate (Corning; 3764). For screening CXCL12 control ligand and test compounds, stock solutions were prepared in assay buffer consisting of Calcium Direct buffer supplemented with 0.5 mg/mL BSA and injection volumes were limited to 2 μL. For antagonist response measurements, assay wells were pre-treated with titrations of antagonist AMD3100 (TOCRIS; 3299) using the TECAN D300e digital dispenser 10 minutes prior to agonist injections. Calcium flux responses were measured on the BMG LABTECH CLARIOstar Plus (BMG LABTECH Inc., Cary, NC) using the CLARIOstar Plus supplier excitation and emission settings for Fluo-4 (Calcium Saturated). Measurements were conducted using a bottom-read kinetic well-mode with measurements occurring at 0.5 sec intervals for 150 sec and injection occurring at 10.0 sec.

### Binding Activity.

Binding activity was measured using the Cisbio Tag-Lite Chemokine CXCR4 system (CisBio, Bedford, MA). This assay was conducted using ready-to-assay Tag-Lite Chemokine CXCR4 labelled cells (CITT1CXCR4), red fluorescent labelled CXCR4 ligand (Cisbio; L0012RED), and 1× Tag-Lite buffer (Cisbio; LABMED) prepared according to supplier guidelines. Compounds and AMD3100 were dispensed into a 384-well microplate (Greiner Bio, Frickenhausen Germany) using the TECAN D300e digital dispenser. CXCR4 Tag-lite cells were thawed and washed in 5 mL 1× Tag-Lite buffer, then re-suspended in 2.7 mL of 1× Tag-Lite buffer. After resuspension, 10 μL of cell suspension was dispensed into each assay well followed by 5 μL of Tag-lite buffer and 5 μL of fluorescent ligand and allowed to incubate at room temperature in a dark place for three hours. Saturation binding and competition binding experiments conducted on the same plate with estimated Kd value of 12.5 nM fluorescent ligand for competition binding experiments. Following three-hour incubation, HTRF measurements were taken using the BMG LABTECH CLARIOstar Plus (BMG LABTECH Inc., Cary, NC) with emission signals set to the Cisbio Tag-Lite Binding Assay supplier guidelines. HTRF ratio calculations completed according to supplier guidelines.

### Animal Studies.

All animal experiments were approved by the Institutional Animal Care and Use Committee at the University of Colorado Denver—Anschutz Medical Campus, and experimental protocols followed the guidelines described in the NIH Guide for the Care and Use of Laboratory Animals. In these experiments, 10 week old, female, genetically diabetic C₅₇BKS.Cg-m/Leprdb/J (Db) mice and heterozygous, non-diabetic (non-Db), age-matched female controls from the Jackson Laboratory (Bar Harbor, ME) were used. Mice were anesthetized with inhaled isoflurane. Each mouse was shaved and depilated before wounding. The dorsal skin was swabbed with alcohol and Betadine (Purdue Pharma, Stamford, CT). Each mouse received a single, full-thickness dorsal wound (including panniculus carnosum) with an 8-mm punch biopsy (Miltex Inc, York, PA). After wounding, a Hamilton syringe was used to deliver 50 μL of either 10 uM test compound or PBS, as a control. 10 μL were injected intradermally at 12, 3, 6 and 9 o'clock and at the wound base. All wounds were dressed with Tegaderm (3 M, St Paul, MN), which was subsequently removed on postoperative day 2. Postoperatively, the mice received a subcutaneous injection of an analgesic, Banamine (Schering-Plough Animal Health Corp., Union, NJ). Mice pictures were taken every other day, wound size was analyzed by Image J. A full-thickness skin sample, centered on the wound, was harvested 3 and 7 days after surgery (n=5 per timepoint).

### Real Time Quantitative PCR

Total RNA was extracted with TRIzol reagent (Invitrogen, Carlsbad, CA, USA) according to the manufacturer's established protocol. RNA was converted into cDNA using the SuperScript First-Strand Synthesis System (Invitrogen, Life Technologies). Primers and probes for mouse miR-15b and miR-29a were acquired from Applied Biosystems TaqMan gene expression assay (Applied Biosystems, Foster City, CA). Quantitative PCR was performed on a BIO-RAD CFX96 according to the manufacturer's instructions. Quantitative values of genes of interest are normalized based on U6. Samples (n=5 per group) were amplified in triplicate and results were averaged for each individual sample. The ΔΔCT method was used to calculate relative gene expression. Results are reported as mean±SD.

### Statistical Analysis and Curve Fitting

Results are expressed as mean±SD for 3 to 5 independent experiments. Statistically significant differences in gene expression between two groups were assessed by Student's T-test, ANOVA with an appropriate post hoc test was be used for multiple comparisons. P<0.05 was considered to be statistically significant. All concentration response curves were analyzed to determine EC₅₀ and Eₘₐₓ using the following equation:

$$Y = \frac{100}{1 + {10}^{( Log EC 50 - X )} \times Hillslope}$$

where Y is the normalized response (0-100%), X is the log of concentration of compound tested, and the Hill slope is equal to 1.0% and 100% activity are specific to each assay. 100% activity for migration data refers to activity induced by EC₉₀ concentration of CXCL12 alone; 0% activity in this assay refers to the activity of wells left untreated with CXCL12 or compounds.

This example illustrates the preparation of thiadiazine scaffold CXCR4 agonists.

Table 2 and Table 3 show exemplary thiadiazole compounds of the present disclosure, including repeated tests; prophetic thiadiazine entries 38, 39, 42, 65, 67, 68, and 72-76. Results for β-arrestin EC₅₀ and Eₘₐₓ values were determined in a β-arrestin recruitment assay normalized to SDF-1.

TABLE 2
Exemplary compounds of Formula IIb

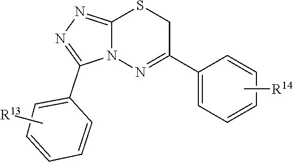

Formula IIb

| | | | TANGO β- | | Migration | |
|---|---|---|---|---|---|---|
| | | | arrestin | | CEM | |
| Thiadiazine | | | EC₅₀ | Eₘₐₓ | EC₅₀ | Eₘₐₓ |
| Entry | R¹³ | R¹⁴ | (μM) | (%) | (μM) | (%) |
| 1 | 2-OMe | 4-OMe | A | B | — | — |
| 2 | 2-OEt | 4-OMe | ND | B | — | — |
| 3 | 2-OEt | 4-OMe | A | A | A | B |
| 4 | 2-OMe | 4-OEt | A | A | A | B |
| 5 | 2-OMe | 4-O-n-Bu | A | C | E | — |
| 6 | 2-OEt | 4-OCH₂CH═CH₂ | A | A | A | B |
| 7 | 2-OEt | 4-OCH₂CH═CH₂ | A | A | A | A |
| 8 | 2-OMe | 4-OCH₂CH₂OCH₃ | A | C | A | C |
| 9 | 2-OEt | 4-OH | A | A | A | A |
| 10 | 2-OEt | 4-OCF₃ | A | A | B | A |
| 11 | 2-OEt | 4-OCHF₂ | A | B | A | A |
| 12 | 2-OMe | 4-SMe | A | A | A | B |
| 13 | 2-OMe | 4-NMe₂ | A | A | A | B |
| 14 | 2-OEt | 4-N(pyrrolidinyl) | A | A | A | B |
| 15 | 2-OEt | 4-N(pyrrolidinyl) | A | A | A | A |
| 16 | 2-OMe | 4-N(morphinyl) | B | A | B | A |
| 17 | 2-OMe | 4-methyl | A | B | — | — |
| 18 | 2-OMe | 4-ethyl | A | B | — | — |
| 19 | 2-OMe | 4-n-Bu | B | C | — | — |
| 20 | 2-OMe | 4-Ph | B | A | — | — |
| 21 | 2-OEt | 4-OMe | A | A | A | B |
| 22 | 2-OMe | 4-NMe₂ | A | A | A | B |
| 23 24 | 2-OEt 2-OEt | 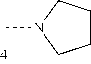 | A A | A A | A A | C A |
| 25 | 2-OMe | 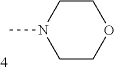 | B | A | — | — |
| 26 | 2-OEt | 4-NH₂ | E | — | — | — |
| 27 | 2-OEt | 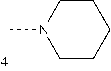 | E | — | E | — |
| 28 | 2-OEt | 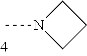 | A | B | A | C |
| 29 | 2-OEt | 1,2,3,4-tetrahydroquinolin- | A | A | B | C |
| | | 6-yl (3,4-fused bicyclic | | | | |
| | | ring) | | | | |
| 30 | 2-OEt | 4-NHCOCH₃ | A | C | E | — |
| 31 | 2-OEt | 4-NHCOCH(CH₃)₂ | E | — | E | — |
| 32 | 2-OEt | 4-NHCOcyclopropyl | A | A | A | C |
| 33 | 2-OEt | 4-NHCOcyclopentyl | E | — | ND | — |
| 34 | 2-OEt | 4-NHCOPh | A | B | A | C |
| 35 | 2-OEt | 4-NHCOPh(2-CO₂H) | E | — | E | — |
| 36 | 2-OEt | 4-NHCOPh(3-CO₂Me) | E | — | E | — |
| 37 | 2-OEt | 4-NHCOPh(4-CO₂Me) | E | — | E | — |
| 38 | 2-OEt | 4-NHCH₂CH₂OCH₃ | — | — | — | — |
| 39 | 2-OEt | 4-NHCH₂CH═CH₂ | — | — | — | — |
| 40 | 2-OEt | 4-NHCH₂COOEt | A | C | B | C |
| 41 | 2-OEt | 4-NHCH₂CH₂COOEt | A | B | A | C |
| 42 | 2-OEt | 4-NHCH₂CH₂COOH | — | — | — | — |
| 43 | 2-OEt | 4-NHCH₂CH₂CH₂C═CH | A | C | — | C |
| 44 | 2-OEt | 4-NH-cyclobutyl | A | A | A | C |
| 45 | 2-OEt | 4-NH-cyclopentyl | A | C | A | C |
| 46 | 2-OEt | 4-NH-cyclohexyl | A | C | A | C |
| 47 | 2-OEt | 4-NHCH₂Ph | E | — | E | — |
| 48 | 2-OEt | 4-NHCH₂cyclopropyl | A | C | E | — |
| 49 | 2-OEt | 4-NHCH₂cyclopentyl | E | — | E | — |
| 50 | H | H | D | ND | — | — |
| 51 | H | 4-OMe | B | B | — | — |
| 52 | H | 3,4-diOMe | D | ND | — | — |
| 53 | 2-OMe | H | D | ND | — | — |
| 54 | 2-OMe | 2,4-diOMe | A | B | — | — |
| 55 | 2-OMe | 4-OMe | A | B | — | — |
| 56 | 2,5-diOMe | 3,4-diOMe | A | B | — | — |
| 57 | 2-OEt | 3,4-diOMe | A | B | — | — |
| 58 | 3-OMe | H | D | ND | — | — |
| 59 | 4-OMe | H | D | ND | — | — |
| 60 | 3-OMe | 4-OMe | A | B | — | — |
| 61 | 3,4,5- | H | D | ND | — | — |
| | triOMe | | | | | |
| 62 | 3,4,5- | 4-Br | A | B | — | — |
| | triOMe | | | | | |
| 63 | 2-OEt | 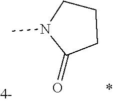 | A | C | A | B |
| 64 | 2-OEt | 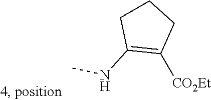 | A | B | A | B |
| 65 | 2-OEt | 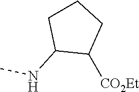 | — | — | — | — |
| 66 | 2-OEt | 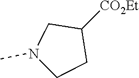 | E | C | A | B |
| 67 | 2-OEt | 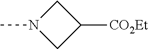 | — | — | — | — |
| 68 | 2-OEt | 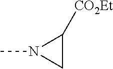 | — | — | — | — |
| 69 | 2-OEt | 4-CONHPh | A | C | A | B |
| 70 | 2-OEt | 4-COOH | E | — | A | B |
| 71 | 2-OEt | 4-COOMe | A | C | A | B |
| 72 | 2-OEt | 4-COOEt | — | — | — | — |
| 73 | 2-OEt | 4-CONHMe | — | — | — | — |
| 74 | 2-OEt | 4-CONHEt | — | — | — | — |
| 75 | 2-OEt | 4-CONMe₂ | — | — | — | — |
| 76 | 2-OEt | 4-CONEt₂ | — | — | — | — |
| 77 | 2-OEt | 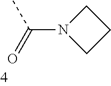 | E | — | A | B |
| 78 | 2-OEt | 4-CONHCH₂COOEt | A | C | E | — |
| 79 | 2-OMe, 5-Cl | 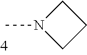 | E | — | E | C |
| 80 | 2,5-dimethoxy | 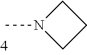 | A | C | A | B |

EC₅₀ (μM) D > 80; 50 < C ≤ 80; 5 < B ≤ 50; A ≤ 5
E_{Max} (%): C ≤ 35; 35 < B < 70; A ≥ 70
E > 10;
ND: not determined
*the corresponding 3-substituted analog is inactive.

TABLE 3
Exemplary compounds of Formula IIc-1

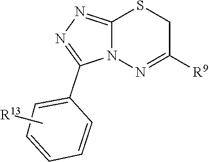

Formula IIc-1

| | | | TANGO β- | | Migration | |
|---|---|---|---|---|---|---|
| Thia- | | | arrestin | | CEM | |
| diazine | | | EC₅₀ | Eₘₐₓ | EC₅₀ | Eₘₐₓ |
| Entry | R¹³ | R⁹ | (mM) | (%) | (mM) | (%) |
| 77 | 2-OEt | 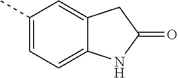 | A | B | A | C |
| 78 | 2-OEt | 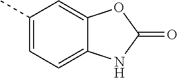 | E | — | E | — |
| 79 | 2-OEt | 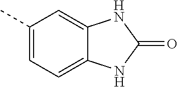 | E | — | E | — |

EC₅₀ (μM) D > 80; 50 < C ≤ 80; 5 < B ≤ 50; A ≤ 5
EMₐₓ (%): C ≤ 35; 35 < B < 70; A ≥ 70
E > 10;
ND: not determined

¹H and ¹³C NMR spectra were recorded on Bruker 500 MHz or 600 MHz spectrometer. Data for 1H NMR (CDCl3 referenced at δ 8.07) and 13C NMR (CDCl3 referenced at δ 77.16) reported as followings: multiplicity (s=singlet, d=doublet, t=triplet, q=quartet, hept=heptet, dd=doublet of doublets, and m=multiplet), integration, coupling constant (Hz), and chemical shift (ppm). All reactions were monitored by thin-layer chromatography (TLC) on silica gel (Merck, 60 Å F-254) TLC plates using UV light as the visualizing agent. Flash column chromatography were performed on silica gel (Merck, 60 Å 0.049-0.063 mm). Building blocks, reagents and starting compounds were purchased from Sigma-Aldrich (St. Louis, MO). Biotage initiator+ was used for microwave accelerated synthesis, Biotage Selekt flash chromatography system adaptable to normal and reverse-phase separation and purification, Biotage V-10 Touch and a Buchi RII rotovap for microscale and larger scale evaporation of solvents and isolation of compounds. NMR analysis was done on AVANCE NEO 600 MHz NMR spectrometer equipped with PRODIGY probe and N2-Cryo-Platform. Low resolution mass spectrometry (LRMS) data were recorded on an instrument by electrospray ionization (ESI) on a Waters Acquity UPLC I-class Core RPHPLC-MS system equipped with PDA and QDA detectors for reaction monitoring and assessing purity of small molecules. All synthesized compounds and commercially purchased compounds that were used in biological screening were >95% in purity as established by RPHPLC-MS.

Synthesis of analogs SP-01-48A (Table 2, Entry 44), SP-01-69 (Table 2, Entry 45), SP-01-70 (Table 2, Entry 46), and SP-01-84 (Table 2, Entry 28):

Starting from 4-amino-5-(2-ethoxyphenyl)-4H-1,2,4-triazole-3-thiol (0.400 g, 1.69 mmol) and tert-butyl (4-(2-chloroacetyl)phenyl)carbamate (0.457 g, 1.69 mmol) using the representative procedure C for the synthesis of compound 36 to obtain 4-(3-(2-ethoxyphenyl)-7H-[1,2,4]triazolo[3,4-b][1,3,4]thiadiazin-6-yl)aniline hydrobromide as a orange-brown solid (200 g, 78%). In a glass vial, 4-(3-(2-ethoxyphenyl)-7H-[1,2,4]triazolol[3,4-b]1[1,3,4]thiadiazin-6-yl)aniline, SP-01-21 (Table 2, Entry 26) was weighed in followed by addition of 1:4 of acetic acid:THF and 5-10 equivalents of the corresponding carbonyl compound, acetal or ketal. After stirring for 10-15 minutes, excess sodium acetoxyborohydride (10 equivalents) was added and mixture stirred overnight. The reaction mixture was extracted with ethyl acetate and organic layer was dried and evaporated and residue was purified by preparative TLC or flash chromatography.

SP-01-48A (Table 2, Entry 44): Starting from SP-01-21 (15 mg, 43 umol) and cyclobutanone (10 uL, 25 equiv.), the product N-cyclobutyl-4-(3-(2-ethoxyphenyl)-7H-[1,2,4]triazolo[3,4-b][1,3,4]thiadiazin-6-yl)aniline, SP-01-48A (5.5 mg) was obtained in 32% yield. ¹H NMR (600 MHz, CDCl₃) δ 7.71-7.66 (m, 1H), 7.67-7.63 (m, 2H), 7.48 (ddd, J=8.4, 7.4, 1.7 Hz, 1H), 7.09 (td, J=7.5, 1.1 Hz, 1H), 6.98 (dd, J=8.4, 1.1 Hz, 1H), 6.55-6.51 (m, 2H), 4.02-3.93 (m, 3H), 3.88 (s, 2H), 2.51-2.42 (m, 2H), 1.94-1.78 (m, 4H), 1.13 (td, J=6.9, 2.2 Hz, 3H). ESI-MS: Calculated for C₂₂H2₃N50S, [M+H]=406.17 and observed [M+H]=406.17.

SP-01-69 (Table 2, Entry 45): Starting from SP-01-21 (22 mg, 63 umol) and cyclobutanone (28 uL, 5 equiv.), the product N-cyclopentyl-4-(3-(2-ethoxyphenyl)-7H-[1,2,4]triazolo[3,4-b][1,3,4]thiadiazin-6-yl)aniline, SP-01-69 (15.3 mg) was obtained in 58% yield. ¹H NMR (600 MHz, CDCl₃) δ 7.69 (dd, J=7.5, 1.8 Hz, 1H), 7.65 (d, J=8.8 Hz, 2H), 7.48 (ddd, J=8.3, 7.5, 1.8 Hz, 1H), 7.09 (td, J=7.5, 1.0 Hz, 1H), 6.98 (dd, J=8.4, 1.0 Hz, 1H), 6.58 (d, J=8.8 Hz, 2H), 3.98 (q, J=7.0 Hz, 2H), 3.88 (s, 2H), 3.85 (s, 1H), 2.12-2.01 (m, 2H), 1.79-1.71 (m, 2H), 1.71-1.63 (m, 2H), 1.54-1.46 (m, 2H), 1.13 (t, J=6.9 Hz, 3H). ESI-MS: Calculated for C₂₃H₂₅N₅OS, [M+H]=420.19 and observed [M+H]=420.20.

SP-01-70 (Table 2, Entry 46): Starting from SP-01-21 (22 mg, 63 umol) and cyclohexanone (30 uL, 5 equiv.), the product N-cyclohexyl-4-(3-(2-ethoxyphenyl)-7H-[1,2,4]triazolo[3,4-b][1,3,4]thiadiazin-6-yl)aniline, SP-01-70 (13.6 mg) was obtained in 55% yield. ¹H NMR (600 MHz, CDCl₃) δ 7.69 (dd, J=7.6, 1.8 Hz, 1H), 7.64 (d, J=8.9 Hz, 1H), 7.48 (ddd, J=8.3, 7.5, 1.8 Hz, 1H), 7.09 (td, J=7.5, 1.0 Hz, 1H), 7.01-6.96 (m, 1H), 6.57 (d, J=8.9 Hz, 1H), 4.06 (s, 1H), 3.98 (q, J=7.0 Hz, 2H), 3.88 (s, 2H), 3.33 (s, 1H), 2.06 (d, J=13.1 Hz, 3H), 1.80 (dt, J=13.6, 3.9 Hz, 2H), 1.72-1.64 (m, 1H), 1.13 (t, J=7.0 Hz, 3H), 0.90 (t, J=7.0 Hz, 2H). ESI-MS: Calculated for C₂₄H₂₇N₅OS, [M+H]=434.20 and observed [M+H]=434.20.

SP-01-84 (Table 2, Entry 28): Starting from SP-01-21 (25 mg, 71 umol) and 1,1,3,3-tetraethoxypropane (68 uL, 4 equiv.), the product 6-(4-(azetidin-1-yl)phenyl)-3-(2-ethoxyphenyl)-7H-[1,2,4]triazolo[3,4-b][1,3,4]thiadiazine, SP-01-84 (12.2 mg) was obtained in 44% yield. ¹H NMR (600 MHz, CDCl₃) δ 7.72-7.64 (m, 3H), 7.48 (ddt, J=10.0, 7.4, 2.0 Hz, 1H), 7.09 (tdd, J=7.4, 2.1, 1.0 Hz, 1H), 6.98 (dt, J=8.5, 1.5 Hz, 1H), 6.68-6.64 (m, 1H), 6.61-6.57 (m, 1H), 4.02-3.94 (m, 2H), 3.88 (s, 2H), 3.75-3.69 (m, 2H), 3.44 (q, J=7.1 Hz, 1H), 3.40-3.34 (m, 1H), 3.23 (t, J=6.8 Hz, 1H), 1.79-1.74 (m, 1H), 1.13 (dt, J=10.4, 7.0 Hz, 3H). ESI-MS: Calculated for C₂₁H₂₁N₅OS, [M+H]=392.15 and observed [M+H]=392.18.

SP-01-29A (Table 2, Entry 30): A 8 mL glass vial was charged with SP-01-21 (25 mg, 71 umol), 4-dimethylaminopyridine (26 mg, 213 umol) and 2 mL acetonitrile. Acetyl chloride (10 uL, 2 equiv.) was added and mixture stirred overnight at room temperature. The reaction mixture was diluted with ethyl acetate (2 mL) and washed with 0.1 M HCl 5-6 times and organic layer collected and evaporated. The residue was purified by preparative TLC (50% ethyl acetate/hexanes) to obtain the product N-(4-(3-(2-ethoxyphenyl)-7H-[1,2,4]triazolo[3,4-b][1,3,4]thiadiazin-6-yl)phenyl)acetamide, SP-01-29A (20 mg, 72%). ¹H NMR (600 MHz, CDCl₃) δ 7.81-7.75 (m, 3H), 7.71-7.65 (m, 3H), 7.50 (ddd, J=8.3, 7.5, 1.8 Hz, 1H), 7.10 (td, J=7.5, 1.0 Hz, 1H), 6.99 (dd, J=8.4, 1.0 Hz, 1H), 4.01-3.92 (m, 4H), 2.23 (s, 3H), 1.11 (t, J=7.0 Hz, 3H). ESI-MS: Calculated for C₂₀H₁₉N₅O₂S, [M+H]=394.13 and observed [M+H]=394.14.

SP-01-38A (Table 2, Entry 31): A 8 mL glass vial was charged with SP-01-21 (15 mg, 43 umol), 4-dimethylaminopyridine (10.4 mg, 85 umol) and 2 mL acetonitrile. Acetyl chloride (7 uL, 1.5 equiv.) was added and mixture stirred overnight at room temperature. The reaction mixture was diluted with ethyl acetate (2 mL) and washed with 0.1 M HCl 5-6 times and organic layer collected and evaporated. The residue was purified by preparative TLC (60% ethyl acetate/hexanes) to obtain the product N-(4-(3-(2-ethoxyphenyl)-7H-[1,2,4]triazolo[3,4-b][1,3,4]thiadiazin-6-yl)phenyl)acetamide, SP-01-38A (11 mg, 64%). ¹H NMR (600 MHz, CDCl₃) 67.83-7.74 (m, 3H), 7.73-7.64 (m, 3H), 7.52-7.46 (m, 1H), 7.10 (tt, J=7.5, 1.1 Hz, 1H), 6.98 (d, J=8.3 Hz, 1H), 3.95 (d, J=8.3 Hz, 4H), 2.58 (pd, J=6.8, 1.0 Hz, 1H), 1.27 (dd, J=6.9, 1.9 Hz, 7H), 1.10 (td, J=7.0, 1.1 Hz, 3H). ESI-MS: Calculated for C₂₂H₂₃N₅O₂S, [M+H]=422.17 and observed [M+H]=422.18.

SP-01-49 (Table 2, Entry 27): Starting from 4-amino-5-(2-ethoxyphenyl)-4H-1,2,4-triazole-3-thiol (25 mg, 106 umol) and 2-bromo-1-(4-(piperidin-1-yl)phenyl)ethan-1-one (30 mg, 106 umol) using the representative procedure for the synthesis of UCUF-965 to obtain 3-(2-ethoxyphenyl)-6-(4-(piperidin-1-yl)phenyl)-7H-[1,2,4]triazolo[3,4-b][1,3,4]thiadiazine (21 mg, 47%). ¹H NMR (600 MHz, CDCl₃) δ 7.69 (tt, J=5.5, 1.8 Hz, 3H), 7.48 (ddd, J=8.8, 5.3, 1.6 Hz, 1H), 7.09 (tdd, J=7.6, 2.0, 1.0 Hz, 1H), 6.98 (d, J=8.3 Hz, 1H), 6.89 (dd, J=9.2, 2.6 Hz, 2H), 3.98 (qd, J=7.0, 1.7 Hz, 2H), 3.90 (d, J=1.7 Hz, 2H), 3.34 (t, J=5.4 Hz, 4H), 1.75-1.62 (m, 6H), 1.13 (td, J=6.9, 2.0 Hz, 3H). ESI-MS: Calculated for C₂₃H₂₅N₅OS, [M+H]=420.19 and observed [M+H]=420.21.

SP-01-176 (Table 2, Entry 40): An 8.0 mL vial under nitrogen was charged with 4-amino-5-(2-ethoxyphenyl)-4H-1,2,4-triazole-3-thiol (50 mg, 142 umol) and 3 mL dry dichloromethane and Rh₂(esp)₂ catalyst (5.4 mg, 5 mol %). Ethyl diazoacetate (60 uL, 3.5 equiv) was added as a dichloromethane solution in 1 equivalent portions followed by stirring at room temperature for 5-10 minutes till no further consumption of starting material was detected. The solvent was evaporated and residue was purified by preparative TLC to obtain ethyl (4-(3-(2-ethoxyphenyl)-7H-[1,2,4]triazolo[3,4-b][1,3,4]thiadiazin-6-yl)phenyl)glycinate, SP-01-176 (34.5 mg, 55%). ¹H NMR (600 MHz, CDCl₃) δ 7.74-7.61 (m, 3H), 7.51-7.45 (m, 1H), 7.09 (tdd, J=7.5, 3.1, 1.0 Hz, 1H), 6.98 (ddd, J=8.3, 4.1, 1.0 Hz, 1H), 6.71-6.58 (m, 2H), 4.80 (s, 1H), 4.26 (dq, J=25.0, 7.2 Hz, 2H), 4.18 (s, 1H), 4.00-3.93 (m, 4H), 3.89 (d, J=3.1 Hz, 2H), 1.31 (dt, J=16.2, 7.1 Hz, 3H), 1.12 (t, J=7.0 Hz, 3H). ESI-MS: Calculated for C₂₂H₂₃N₅O₃S, [M+H]=438.16 and observed [M+H]=438.13.

SP-01-182 (Table 4, Entry 40a): A 8.0 mL glass vial was charged with SP-01-176 (14 mg, 32 umol) and Lithium hydroxide monohydrate (4 mg, 96 umol) followed by addition of 0.5 mL ethanol and 2 mL water. The mixture was stirred overnight and diluted with 2 mL ethyl acetate and 1 mL water. The organic layer was separated, dried and evaporated to obtain the product (4-(3-(2-ethoxyphenyl)-7H-[1,2,4]triazolo[3,4-b][1,3,4]thiadiazin-6-yl)phenyl)glycine, SP-01-182 (11 mg, 84%). ¹H NMR (600 MHz, MeOD) δ 7.75-7.69 (m, 2H), 7.58-7.51 (m, 2H), 7.17-7.07 (m, 2H), 6.70-6.57 (m, 2H), 4.24 (s, 1H), 4.20-4.15 (m, 2H), 4.01 (q, J=7.0 Hz, 2H), 3.94 (s, 1H), 1.12 (td, J=7.0, 2.2 Hz, 3H). ESI-MS: Calculated for C₂₀H₁₉N₅O₃S, [M+H]=410.13 and observed [M+H]=410.14.

SP-01-110 (Table 2, Entry 63): Starting from 4-amino-5-(2-ethoxyphenyl)-4H-1,2,4-triazole-3-thiol (50 mg, 212 mmol) and 1-(4-(2-bromoacetyl)phenyl)pyrrolidin-2-one (60 mg, 212 mmol) using the representative procedure for the synthesis of UCUF-965 to obtain 1-(4-(3-(2-ethoxyphenyl)-7H-[1,2,4]triazolo[3,4-b][1,3,4]thiadiazin-6-yl)phenyl)pyrrolidin-2-one, SP-01-110 (74 mg, 83%). ¹H NMR (600 MHz, CDCl₃) δ 7.83-7.79 (m, 2H), 7.78-7.73 (m, 2H), 7.68 (dd, J=7.5, 1.8 Hz, 1H), 7.48 (ddd, J=8.2, 7.4, 1.8 Hz, 1H), 7.09 (td, J=7.5, 1.0 Hz, 1H), 6.97 (dd, J=8.4, 1.0 Hz, 1H), 3.98-3.92 (m, 5H), 3.89 (t, J=7.1 Hz, 2H), 2.65 (t, J=8.1 Hz, 2H), 2.24-2.16 (m, 2H), 1.09 (t, J=7.0 Hz, 3H). ESI-MS: Calculated for C₂₂H₂₁N₅O₂S, [IM+H]=420.15 and observed [IM+H]=420.20.

### Example 2

Table 4 reports select Thiadiazine compounds and compounds in Entries 1, 3, 17, 18, 50, 51, 53, 54, 57, 60, and 81-90 were procured from commercial sources including ChemDiv, San Diego, USA and Princeton Biomolecular, Princeton, New Jersey, USA. Purity was established to be >95% by LCMS and powders were used as is for biological screening.

TABLE 4
Activity of select Thiadiazine compounds of Formula IIb

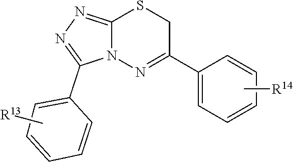

Formula IIb

| | | | ß-arrestin | | | | Migration | |
|---|---|---|---|---|---|---|---|---|
| Thia- | | | recruitment | | cAMP | | CEM | |
| diazine | | | EC₅₀ | Eₘₐₓ | EC₅₀ | Eₘₐₓ | EC₅₀ | Eₘₐₓ |
| Entry | R¹³ | R¹⁴ | (μM) | (%) | (μM) | (%) | (μM) | (%) |
| 1 | 2-OMe | 4-OMe | 0.054 | 45 | | | | |
| 50 | H | H | >80 | NA | | | | |
| 53 | 2-OMe | H | >80 | NA | | | | |
| 51 | H | 4-OMe | 11.2 | 54 | | | | |
| 81 | 2-OMe | 4-fluoro | >80 | NA | | | | |
| 82 | 2-OMe | 4-chloro | 4.14 | 44 | | | | |
| 83 | 2-OMe | 4-bromo | 1.51 | 37 | | | | |
| 84 | 2-OMe | 4-nitro | >80 | NA | | | | |
| 17 | 2-OMe | 4-methyl | 0.268 | 42 | | | | |
| 18 | 2-OMe | 4-ethyl | 0.203 | 44 | | | | |
| 19 | 2-OMe | 4-n-Bu | 14.1 | 12 | | | | |
| 20 | 2-OMe | 4-Ph | 8.35 | 95 | | | | |
| 85 | 2-OMe | 3-OMe | 10.7 | 40 | | | | |
| 86 | 2-OMe | 2-OMe | >80 | NA | | | | |
| 54 | 2-OMe | 2,4- | 0.866 | 23 | | | | |
| | | dimethoxy | | | | | | |
| 60 | 3-OMe | 4-OMe | 2.51 | 63 | | | | |
| 87 | 4-OMe | 4-OMe | 6.71 | 65 | | | | |
| 88 | 2-Cl | 4-OMe | 0.282 | 43 | | | | |
| 89 | 3-Cl | 4-OMe | >80 | NA | | | | |
| 90 | 4-Cl | 4-OMe | 7.75 | 44 | | | | |
| 3 | 2-OEt | 4-OMe | 0.13 | 60 | | | | |
| 57 | 2-OEt | 3,4- | 0.571 | 42 | | | | |
| | | dimethoxy | | | | | | |
| 9 | 2-OEt | 4-OH | 1.28 | 96 | | | | |
| 5 | 2-OMe | 4-O-n-Bu | 0.7 | 31.3 | | | | |
| 6 | 2-OEt | 4-OCH₂ | 0.20 | 95 | | | | |
| | | CH═CH₂ | | | | | | |
| 8 | 2-OMe | 4-OCH₂ | 3.43 | 28 | | | | |
| | | CH₂OCH₃ | | | | | | |
| 10 | 2-OEt | 4-OCF₃ | 1.76 | 83 | | | | |
| 11 | 2-OEt | 4-OCHF₂ | 0.3 | 48 | | | | |
| 13 | 2-OMe | 4-NMe₂ | 0.06 | 91 | | | | |
| 44 | 2-OEt | 4-NH- | 0.33 | 77 | 0.4 | 19 | 0.2 | 16 |
| | | cyclobutyl | | | | | | |
| 45 | 2-OEt | 4-NH- | 1.4 | 31 | | | | |
| | | cyclo- | | | | | | |
| | | pentyl | | | | | | |
| 46 | 2-OEt | 4-NH- | 0.77 | 10 | | | | |
| | | cyclo- | | | | | | |
| | | hexyl | | | | | | |
| 26 | 2-OEt | 4-NH₂ | >10 | NA | | | | |
| 30 | 2-OEt | 4- | >10 | NA | | | | |
| | | NHCOCH₃ | | | | | | |
| 31 | 2-OEt | 4- | >10 | NA | | | | |
| | | NHCOCH | | | | | | |
| | | (CH₃)₂ | | | | | | |
| 23 | 2-OEt | 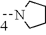 | 0.02 | 44 | 0.05 | 19 | 0.40 | 31 |
| 28 | 2-OEt | 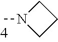 | 0.10 | 40 | 0.16 | 22 | 0.18 | 12 |
| 27 | 2-OEt | 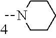 | >10 | NA | | | | |
| 25 | 2-OMe | 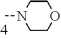 | 7.81 | 96 | | | | |
| 40 | 2-OEt | 4-NHCH₂ | >5 | | | | | |
| | | COOEt | | | | | | |
| 40a | 2-OEt | 4-NHCH₂ | 0.03 | 43 | 1.6 | 17 | 0.32 | 23 |
| | | COOH | | | | | | |
| 63 | 2-OEt | 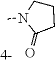 | 0.05 | 22 | 0.08 | 18 | 0.22 | 32 |

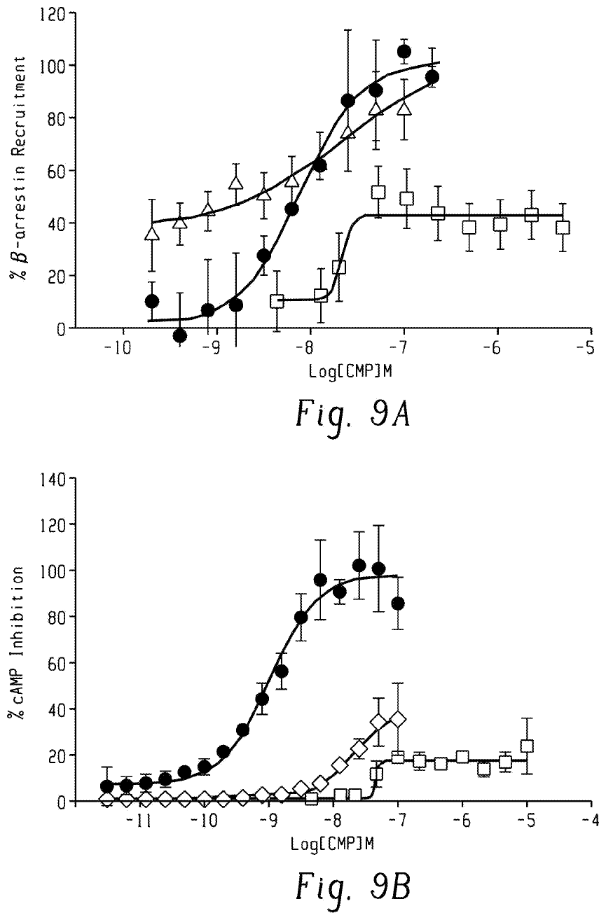

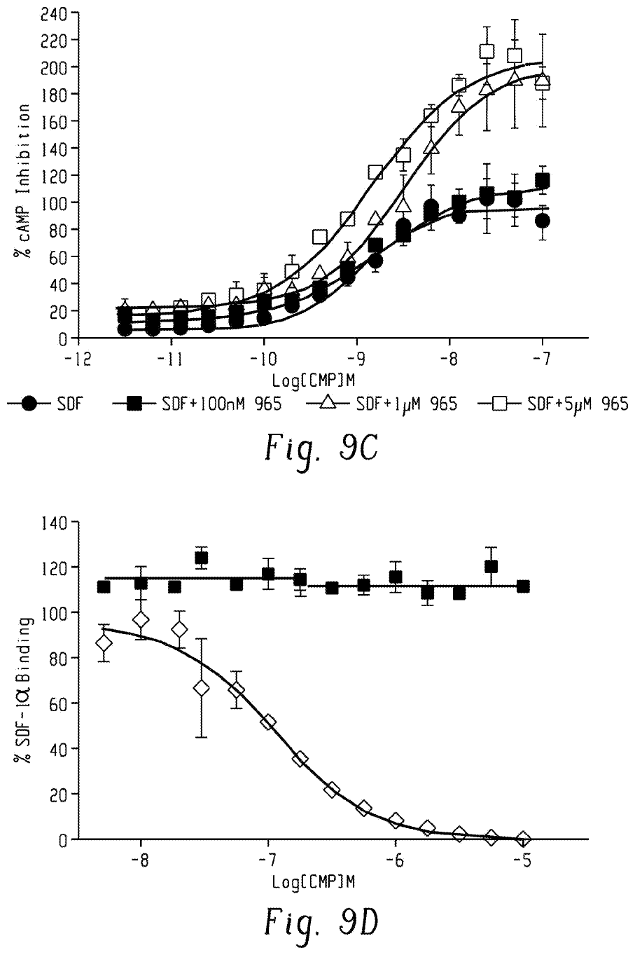

Among the compounds of Table 4, UCUF-965 (Table 4, Entry 23) is the most potent among the small molecule agonists of CXCR4/SDF-1α signaling for β-arrestin recruitment with an EC₅₀=0.02 μM (log −7.7±0.1 M, n=3) and average Eₘₐₓ=44% normalized to maximal SDF-1α response (FIG. 9A).

SDF-1α potently inhibited forskolin-stimulated cAMP production by adenylate cyclase in CXCR4 overexpressing CHO cells as a measure of its activity on CXCR4 mediated G-protein coupled signaling (Gᵢ) pathway (IC₅₀=1.5 nM, −8.9±0.1 M) and this response was attenuated in the presence of 1 μM AMD3100 with 20-fold rightward shift in IC₅₀ and reduction of Eₘₐₓ to 40% (FIG. 9B). UCUF-965 was evaluated in a cAMP assay normalized to the maximal response of SDF-1α and the compound showed dose-dependent partial agonist response with EC₅₀=0.05 μM (log −7.3±0.2 M, n=3) and average Eₘₐₓ=19% of cAMP inhibition. Pretreatment with increasing concentrations of UCUF-965 of 0.1, 1, and 5 μM did not significantly affect the EC₅₀ value of SDF-1α alone; however, in the presence of the compound the cAMP maximal response (Eₘₐₓ) of SDF-1α was potentiated by 110, 201, and 209%, respectively. UCUF-965 showed saturation at 5 μM concentration of activator, indicative of allosteric activation of CXCR4/SDF-1α signaling by UCUF-965. To assess binding to the target CXCR4 receptor, UCUF-965 was tested in a competitive binding assay in Tag-Lite Chemokine CXCR4 labelled cells (FIG. 9C). UCUF-965 did not displace fluorescently labelled SDF-1α binding at a constant concentration of 12.5 nM, whereas AMD3100 dose-dependently displaced SDF-1α with an IC₅₀=110 nM, a potency comparable to literature values. These results confirm that UCUF-965 does not bind orthosterically to the SDF-1α site on the CXCR4 receptor.

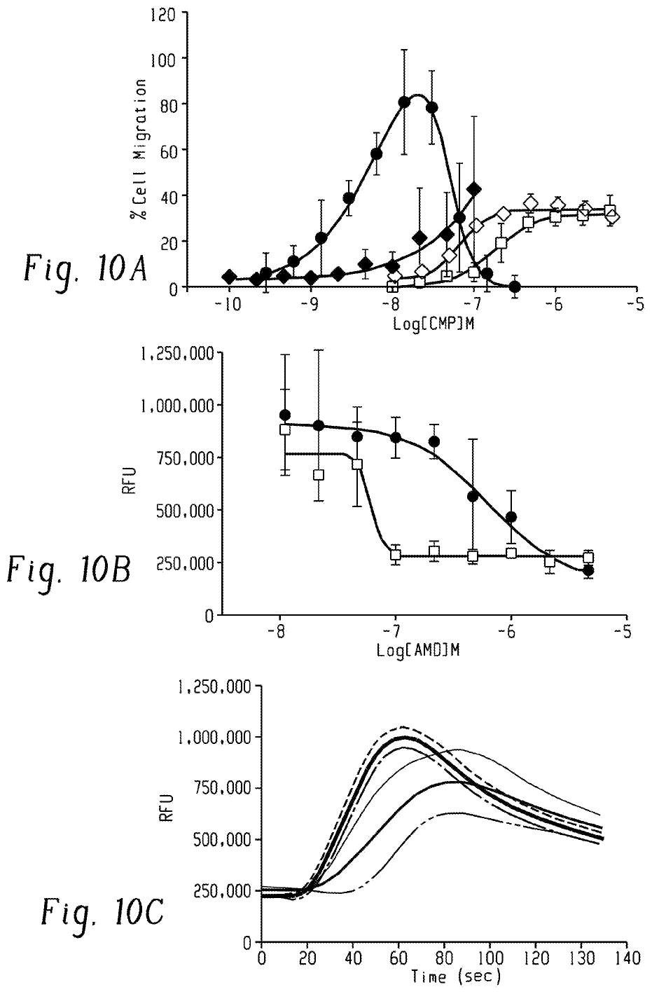

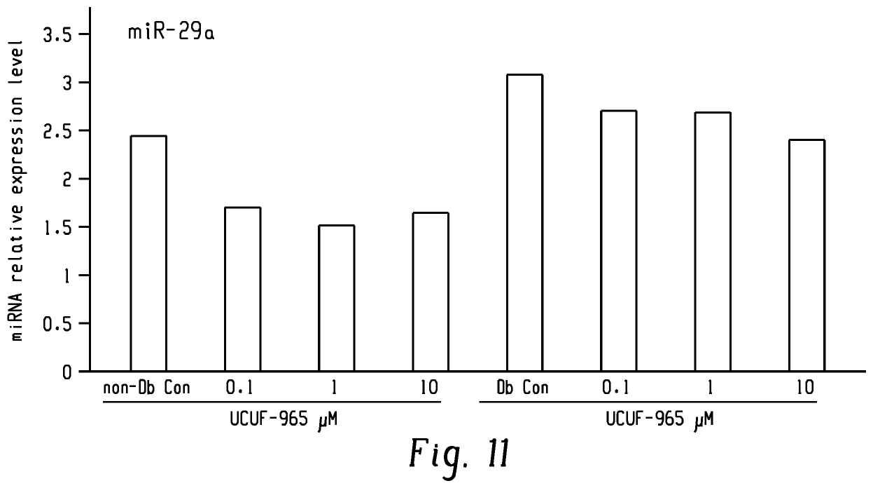

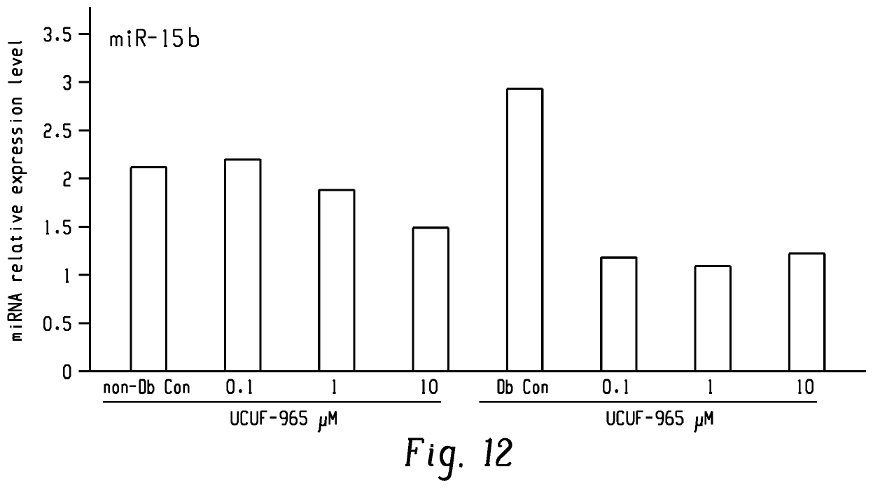

SDF-1α stimulates chemotaxis in a high percentage of resting and active T lymphocytes, and the CXCR4 receptor is highly expressed in the CEM (Leukemia) cell line. To confirm functional activity, UCUF-965 was evaluated in a transwell migration assay utilizing CEM-CCRF human lymphoblast cells (FIG. 10A). UCUF-965 induced migration at EC₅₀=0.4 μM (log −6.4±0.3 M, n=3) and average Eₘₐₓ=31% indicating 10-fold lower potency in the endogenous assay compared to the compound's activity in the β-arrestin recruitment assay. AMD3100 completely blocks SDF-1α induced migration as previously reported. AMD3100 was unable to inhibit UCUF-965-induced migration. Further, In CEM-CCRF cells, 10 μM UCUF-965 induces CXCR4-mediated calcium flux in comparison to 10 nM SDF-1α (FIG. 10C). Pre-treatment with AMD 3100 resulted in dose-dependent inhibition of SDF-1α-induced calcium mobilization at an IC₅₀=630 nM (FIG. 10B). In the case of downstream calcium mobilization, AMD 3100 also dose-dependently inhibited UCUF-965 mediated activation at 10-fold higher potency (IC₅₀=60 nM, FIG. 10B) reflecting that UCUF-965 is a weaker agonist compared with the endogenous CXCR4 ligand. This lends further proof for mode of action of UCUF-965 as a partial agonist and a potential allosteric modulator of CXCR4 signaling.

Four active analogs of UCUF-965 (Table 4 Entries 44, 28, 40a, 63) were studied for inhibition of forskolin stimulated cAMP production and stimulation of migration of CEM-CCRF human lymphoblast cells. All four compounds showed partial agonist activity in both the cAMP and migration assays like UCUF-965.

### Ex Vivo Validation of UCUF-965 in Modulating Levels of Micro-RNA:

The expression of specific micro-RNA levels reported to be crucial in the wound-healing process were measured in order to validate and correlate the in vitro activity of UCUF-965 as a selective CXCR4 receptor modulator to micro-RNA expression. Murine diabetic and non-diabetic fibroblasts were cultured with increasing concentrations of the compound. The cells were incubated for 24 h and then total cellular RNA was isolated to examine the ability of the compound to correct the abnormal expression levels of miR-15b, miR-29a, and miR-146a. The effect of UCUF-965 on expression of miR-15b, which inhibits angiogenesis and wound repair was examined. Previous studies show that diabetic wounds have increased miR-15b expression at the baseline compared to non-diabetic skin. UCUF-965 treatment decreased the expression of miR-15b in both diabetic and nondiabetic fibroblasts in a dose dependent manner. In preliminary studies, it was shown that miR-29a and miR-146a, which control collagen production and pro-inflammatory pathways, respectively are significantly dysregulated in diabetic wounds. UCUF-965 resulted in a significant decrease of miR-29a levels and increase of miR-146a levels.

### UCUF-965 Enhances Angiogenesis in Diabetic Wounds:

The effect of UCUF-965 treatment on angiogenesis was assessed using immunohistochemistry for the endothelial marker CD31. Photos of immunoperoxidase staining for CD31 at 7 days in non-diabetic control wounds treated with PBS and diabetic wounds treated with PBS or treated with UCUF-965 were obtained. Quantification of the staining was obtained. The results support that diabetic wounds treated with PBS have significantly less CD31 positive cells per high power field compared to non-diabetic wounds treated with PBS. In contrast, treatment with UCUF-965 significantly increased the number of vessels compared with PBS-treated diabetic wounds suggesting that UCUF-965 stimulates angiogenesis in the wound healing model. Diabetic wounds treated with UCFU-965 demonstrated increased staining of CD31+cells compared to diabetic wounds treated with PBS (P<0.05).

### Terminology

Compounds are described using standard nomenclature. Unless defined otherwise, all technical and scientific terms used herein have the same meaning as is commonly understood by one of skill in the art to which this invention belongs.

The terms “a” and “an” do not denote a limitation of quantity, but rather denote the presence of at least one of the referenced items. The term “or” means “and/or.” The terms “comprising,” “having,” “including,” and “containing” are to be construed as open-ended terms (i.e., meaning “including, but not limited to”). The opened ended term “comprising” includes the intermediate and closed terms “consisting essentially of” and “consisting of.”

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in any suitable order unless otherwise indicated herein or otherwise clearly contradicted by context. The use of any and all examples, or exemplary language (e.g., “such as”), is intended for illustration and does not pose a limitation on the scope of the disclosure unless otherwise claimed. No language in the specification should be construed as indicating any non-claimed element as essential to the practice of the invention. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

All compounds are understood to include all possible isotopes of atoms occurring in the compounds. Isotopes include those atoms having the same atomic number but different mass numbers. By way of general example, and without limitation, isotopes of hydrogen include tritium and deuterium and isotopes of carbon include ¹¹C, ¹³C, and ¹⁴C.

The term “substituted” means that any one or more hydrogens on the designated atom or group is replaced with a selection from the indicated group, provided that the designated atom's normal valence is not exceeded. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent.

A dash (“-”) that is not between two letters or symbols is used to indicate a point of attachment for a substituent.

“Alkyl” includes both branched and straight chain saturated aliphatic hydrocarbon groups, having the specified number of carbon atoms, generally from 1 to about 8 carbon atoms. The term C₁-C₆alkyl as used herein indicates an alkyl group having from 1, 2, 3, 4, 5, or 6 carbon atoms. Other embodiments include alkyl groups having from 1 to 8 carbon atoms, 1 to 4 carbon atoms or 1 or 2 carbon atoms, e.g. C₁-C₅alkyl, C₁-C₄alkyl, and C₁-C₂alkyl. When C₀-Cₙ alkyl is used herein in conjunction with another group, for example, —C₀-C₂alkyl(phenyl), the indicated group, in this case phenyl, is either directly bound by a single covalent bond (C₀alkyl), or attached by an alkyl chain having the specified number of carbon atoms, in this case 1, 2, 3, or 4 carbon atoms. Alkyls can also be attached via other groups such as heteroatoms as in—O—C₀-C₄alkyl(C₃-C₇cycloalkyl). Examples of alkyl include, but are not limited to, methyl, ethyl, n-propyl, isopropyl, n-butyl, 3-methylbutyl, t-butyl, n-pentyl, and sec-pentyl.

“Alkenyl” is a branched or straight chain aliphatic hydrocarbon group having one or more carbon-carbon double bonds that may occur at any stable point along the chain, having the specified number of carbon atoms. Examples of alkenyl include, but are not limited to, ethenyl and propenyl.

“Alkynyl” is a branched or straight chain aliphatic hydrocarbon group having one or more double carbon-carbon triple bonds that may occur at any stable point along the chain, having the specified number of carbon atoms.

“Alkoxy” is an alkyl group as defined above with the indicated number of carbon atoms covalently bound to the group it substitutes by an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy.

“Cycloalkyl” is a saturated hydrocarbon ring group, having the specified number of carbon atoms, usually from 3 to about 7 carbon atoms. Examples of cycloalkyl groups include cyclopropyl, cyclobutyl, cyclopentyl, or cyclohexyl as well as bridged or caged saturated ring groups such as norbornane or adamantane. “—(C₀-Cₙalkyl)cycloalkyl” is a cycloalkyl group attached to the position it substitutes either by a single covalent bond (C₀) or by an alkylene linker having 1 to n carbon atoms.

“Halo” or “halogen” means fluoro, chloro, bromo, or iodo.

“Oxo” means —CO— or

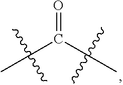 [SMILES: *C(*)=O]

where [symbol] indicates a point of attachment to the adjacent functional group.

The term “aryl” or “aromatic”, in context, refers to a substituted (as otherwise described herein) or unsubstituted monovalent aromatic radical (e.g., a 5-16 membered ring) having a single ring (e.g., benzene, phenyl, benzyl, or 5, 6, 7 or 8 membered ring) or condensed rings (e.g., naphthyl, anthracenyl, phenanthrenyl, 10-16 membered ring, etc.) and can be bound to the compound according to the present disclosure at any available stable position on the ring(s) or as otherwise indicated in the chemical structure presented. Other examples of aryl groups, in context, may include heterocyclic aromatic ring systems, “heteroaryl” groups having one or more nitrogen, oxygen, or sulfur atoms in the ring (moncyclic) such as imidazole, furyl, pyrrole, furanyl, thiene, thiazole, pyridine, pyrimidine, pyrazine, triazole, oxazole or fused ring systems such as indole, quinoline, indolizine, azaindolizine, benzofurazan, etc., among others, which may be optionally substituted as described above. Among the heteroaryl groups which may be mentioned include nitrogen-containing heteroaryl groups such as pyrrole, pyridine, pyridone, pyridazine, pyrimidine, pyrazine, pyrazole, imidazole, triazole, triazine, tetrazole, indole, isoindole, indolizine, azaindolizine, purine, indazole, quinoline, dihydroquinoline, tetrahydroquinoline, isoquinoline, dihydroisoquinoline, tetrahydroisoquinoline, quinolizine, phthalazine, naphthyridine, quinoxaline, quinazoline, cinnoline, pteridine, imidazopyridine, imidazotriazine, pyrazinopyridazine, acridine, phenanthridine, carbazole, carbazoline, pyrimidine, phenanthroline, phenacene, oxadiazole, benzimidazole, pyrrolopyridine, pyrrolopyrimidine and pyridopyrimidine; sulfur-containing aromatic heterocycles such as thiophene and benzothiophene; oxygen-containing aromatic heterocycles such as furan, pyran, cyclopentapyran, benzofuran and isobenzofuran; and aromatic heterocycles comprising 2 or more hetero atoms selected from among nitrogen, sulfur and oxygen, such as thiazole, thiadizole, isothiazole, benzoxazole, benzothiazole, benzothiadiazole, phenothiazine, isoxazole, furazan, phenoxazine, pyrazoloxazole, imidazothiazole, thienofuran, furopyrrole, pyridoxazine, furopyridine, furopyrimidine, thienopyrimidine and oxazole, among others, all of which may be optionally substituted.

“Heteroaryl” is a stable monocyclic aromatic ring having the indicated number of ring atoms which contains from 1 to 3, or in some embodiments from 1 to 2, heteroatoms chosen from N, O, and S, with remaining ring atoms being carbon, or a stable bicyclic or tricyclic system containing at least one 5- to 7-membered aromatic ring which contains from 1 to 3, or in some embodiments from 1 to 2, heteroatoms chosen from N, O, and S, with remaining ring atoms being carbon. Monocyclic heteroaryl groups can have from 5 to 7 ring atoms. In some embodiments bicyclic heteroaryl groups are 9- to 10-membered heteroaryl groups, that is, groups containing 9 or 10 ring atoms in which one 5- to 7-member aromatic ring is fused to a second aromatic or non-aromatic ring. When the total number of S and O atoms in the heteroaryl group exceeds 1, these heteroatoms are not adjacent to one another. In an embodiment, the total number of S and O atoms in the heteroaryl group is not more than 2. In another embodiment, the total number of S and O atoms in the aromatic heterocycle is not more than 1. Heteroaryl groups include, but are not limited to, oxazolyl, piperazinyl, pyranyl, pyrazinyl, pyrazolopyrimidinyl, pyrazolyl, pyridizinyl, pyridyl, pyrimidinyl, pyrrolyl, quinolinyl, tetrazolyl, thiazolyl, thienylpyrazolyl, thiophenyl, triazolyl, benzo[d]oxazolyl, benzofuranyl, benzothiazolyl, benzothiophenyl, benzoxadiazolyl, dihydrobenzodioxynyl, furanyl, imidazolyl, indolyl, isothiazolyl, and isoxazolyl.

“Heterocycle” is a saturated, unsaturated, or aromatic cyclic group having the indicated number of ring atoms containing from 1 to about 3 heteroatoms chosen from N, O, and S, with remaining ring atoms being carbon. Examples of heterocycle groups include piperazine and thiazole groups. Exemplary heterocyclics include: azetidinyl, benzimidazolyl, 1,4-benzodioxanyl, 1,3-benzodioxolyl, benzoxazolyl, benzothiazolyl, benzothienyl, dihydroinidazolyl, dihydropyranyl, dihydrofuranyl, dioxanyl, dioxolanyl, ethyleneurea, 1,3-dioxolane, 1,3-dioxane, 1,4-dioxane, furyl, homopiperidinyl, inidazolyl, imidazolinyl, imidazolidinyl, indolinyl, indolyl, isoquinolinyl, isothiazolidinyl, isothiazolyl, isoxazolidinyl, isoxazolyl, morpholinyl, naphthyridinyl, oxazolidinyl, oxazolyl, pyridone, 2-pyrrolidone, pyridine, piperazinyl, N-methylpiperazinyl, piperidinyl, phthalimide, succinimide, pyrazinyl, pyrazolinyl, pyridyl, pyrimidinyl, pyrrolidinyl, pyrrolinyl, pyrrolyl, quinolinyl, tetrahydrofuranyl, tetrahydropyranyl, tetrahydroquinoline, thiazolidinyl, thiazolyl, thienyl, tetrahydrothiophene, oxane, oxetanyl, oxathiolanyl, thiane among others

“Heterocycloalkyl” is a saturated cyclic group having the indicated number of ring atoms containing from 1 to about 3 heteroatoms chosen from N, O, and S, with remaining ring atoms being carbon. Examples of heterocycloalkyl groups include tetrahydrofuranyl and pyrrolidinyl groups.

“Haloalkyl” means both branched and straight-chain alkyl groups having the specified number of carbon atoms, substituted with 1 or more halogen atoms, generally up to the maximum allowable number of halogen atoms. Examples of haloalkyl include, but are not limited to, trifluoromethyl, difluoromethyl, 2-fluoroethyl, and penta-fluoroethyl.

“Haloalkoxy” is a haloalkyl group as defined above attached through an oxygen bridge (oxygen of an alcohol radical).

“Carboxyl” denotes the group —C(O)OR, where R is hydrogen, alkyl, substituted alkyl, aryl, substituted aryl, heteroaryl or substituted heteroaryl, whereas these generic substituents have meanings which are identical with definitions of the corresponding groups defined herein.

The term “hydrocarbyl” shall mean a compound which contains carbon and hydrogen and which may be fully saturated, partially unsaturated or aromatic and includes aryl groups, alkyl groups, alkenyl groups and alkynyl groups.

The term “independently” is used herein to indicate that the variable, which is independently applied, varies independently from application to application.

The term “pharmaceutically acceptable salt”, as used herein, includes derivatives of the disclosed compounds in which the parent compound is modified by making inorganic and organic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are used, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Lists of additional suitable salts may be found, e.g., in Remington's Pharmaceutical Sciences, 17th ed., Mack Publishing Company, Easton, Pa., p. 1418 (1985).

A “protein” means sequence of peptides having a length of 6 or more, 20 or more, or 50 or more, or 100 or more peptides. The peptides can be natural, synthetic, or semisynthetic. The protein can be natural, synthetic, or semisynthetic, and can be modified from its natural state.

A “patient” means a human or non-human animal in need of medical treatment. Medical treatment can include treatment of an existing condition, such as a disease or disorder, preventative treatment, or diagnostic treatment. In some embodiments the patient is a human patient.

The term “carrier”, as used herein, applied to pharmaceutical compositions refers to a diluent, excipient, or vehicle with which an active compound is provided.

As used herein, “promoting wound healing” means treating a subject with a wound and achieving healing, either partially or fully, of the wound. Promoting wound healing can mean, e.g., one or more of the following: promoting epidermal closure; promoting migration of the dermis; promoting dermal closure in the dermis; reducing wound healing complications, e.g., hyperplasia of the epidermis and adhesions; reducing wound dehiscence; and promoting proper scab formation.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A compound of Formula I 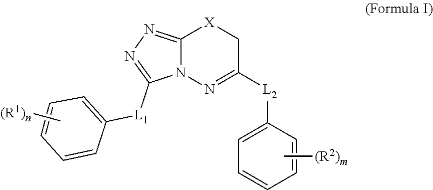
or a pharmaceutically acceptable salt thereof, wherein in Formula I,
R¹ is each independently halogen, cyano, nitro, NR³R⁴, —C₁-C₆alkyl, —C₁-C₆haloalkyl, —C₂-C₆alkenyl, —C₂-C₆alkynyl, —C₁-C₆alkoxy, —C₀-C₆alkylCOOR⁵, —C₀-C₆alkyl(C₃-C₇cycloalkyl), —C₀-C₆alkyl(heterocycloalkyl), —C₀-C₆alkyl(aryl), or —C₀-C₆alkyl(heteroaryl), wherein groups except hydrogen, halogen, cyano, and nitro are optionally substituted with halogen, cyano, nitro, oxo (C═O), a —C₁-C₆alkyl, a —C₃-C₇cycloalkyl, a heterocycloalkyl, a heteroaryl, or an aryl;
R² is each independently halogen, cyano, nitro, —C₁-C₆alkyl, —C₂-C₆alkenyl, —C₂-C₆alkynyl, —C₁-C₆alkoxy, —C₀-C₆alkylCOR⁵, —C₀-C₆alkylCOOR⁵, —C₀-C₆alkyl(C₃-C₇cycloalkyl), —C₀-C₆alkyl(heterocycloalkyl), —C₀-C₆alkyl(aryl), or —C₀-C₆alkyl(heteroaryl), —NR³C₀-C₆alkylCOR⁵, or NR³R⁴, wherein groups except hydrogen, halogen, cyano, and nitro are optionally substituted with halogen, cyano, nitro, oxo (C═O), a —C₁-C₆alkyl, —C₁-C₆haloalkyl, a —C₃-C₇cycloalkyl, a heterocycloalkyl, a heteroaryl, or an aryl, and wherein at least one R² is NR³R⁴ or —NR³C₀-C₆alkylCOR⁵;
or wherein two R²s along with the C to which they are attached form a cyclic ring of 4 to 7 ring atoms or bicyclic ring of 8 to 10 ring atoms, the cyclic or bicyclic ring having 1, 2, or 3 ring atoms independently chosen from N, O, and S, wherein the cyclic or bicyclic ring is substituted with 0-2 substituents independently chosen from halogen, oxo, cyano, C₁-C₆alkyl, C₁-C₆alkoxy, C₁-C₆haloalkyl, and C₁-C₆haloalkoxy;
X is CH₂, O, NRS, or S;
L₁ and L₂ are each independently a bond (absent) or —C₁-C₂₀hydrocarbyl;
R³ and R⁴ are each independently hydrogen, —C₁-C₆alkyl, —C₂-C₆alkenyl, —C₂-C₆alkynyl, —C₀-C₆alkyl-C₁-C₆alkoxy, —C₀-C₆alkylCOR⁶, —C₀-C₆alkyl-COOR⁶, —C₀-C₆alkyl-C₂-C₆alkenyl, C₀-C₆alkyl-C₂-C₆alkynyl, —C₀-C₆alkyl(aryl), or —C₀-C₆alkyl(heteroaryl), wherein groups except hydrogen are optionally substituted with halogen, cyano, nitro, oxo, —C₀-C₆alkylCOOR⁷, —C₁-C₆alkyl, —C₁-C₆haloalkyl, —C₃-C₇cycloalkyl, a heterocycloalkyl, a heteroaryl, or an aryl, and wherein at least one of R³ and R⁴ is not hydrogen;
or R³ and R⁴ along with the N form a cyclic ring of 4 to 7 ring atoms or bicyclic heterocyclic ring of 8 to 10 ring atoms, the cyclic or bicyclic ring having 1, 2, or 3 ring atoms independently chosen from N, O, and S, wherein the cyclic or bicyclic heterocyclic ring is substituted with 0-2 substituents independently chosen from halogen, cyano, oxo, C₁-C₆alkyl, C₁-C₆alkoxy, C₁-C₆haloalkyl, and C₁-C₆haloalkoxy;
R⁵, R⁶, and R⁷ are each independently hydrogen, —C₁-C₆alkyl, —C₁-C₆haloalkyl, —C₀-C₆alkyl(C₃-C₇cycloalkyl), —C₁-C₆alkyl-C₁-C₆alkoxy, —C₁-C₆alkyl-C₂-C₆alkenyl, or C₁-C₆alkyl-C₂-C₆alkynyl; and
n and m are each independently an integer of 1, 2, 3, 4, or 5.

2. The compound of claim 1, wherein
R¹ is —C₁-C₆alkoxy,
R² is NR³R⁴;
R³ and R⁴ are each independently hydrogen, —C₁-C₆alkyl-C₁-C₆alkoxy, —C₁-C₆alkyl-COOR⁵, or C₁-C₆alkyl-C₂-C₆alkynyl;
X is S;
L₁ and L₂ are a bond; and
n and m are each independently is an integer of 1, 2, 3, 4, or 5.

3. The compound of claim 1, where the compound is of Formula Ia 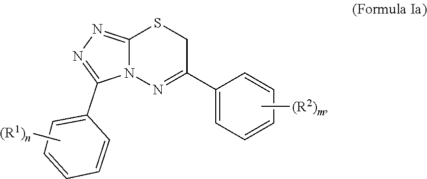
or a pharmaceutically acceptable salt thereof.

4. The compound of claim 3, wherein
R¹ is —C₁-C₆alkoxy,
R² is NR³R⁴;
R³ and R⁴ are each independently hydrogen, —C₁-C₆alkyl-C₁-C₆alkoxy, —C₁-C₆alkyl-COOR⁵, or C₁-C₆alkyl-C₂-C₆alkynyl; and
n and m are each independently is an integer of 1, 2, or 3.

5. The compound of claim 1, where the compound is of Formula Ib 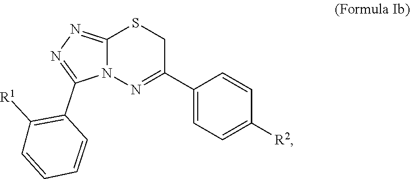
or a pharmaceutically acceptable salt thereof.

6. The compound of claim 5, wherein
R¹ is −C₁-C₆alkoxy,
R² is NR³R⁴; and
R³ and R⁴ are each independently hydrogen, —C₁-C₆alkyl-C₁-C₆alkoxy, —C₁-C₆alkyl-COOR⁵, or C₁-C₀alkyl-C₂-C₆alkynyl.

7. The compound of claim 1, where the compound is 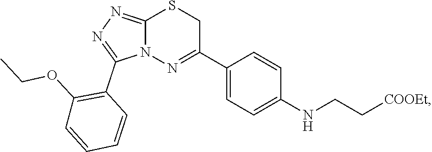 [SMILES: CCOC(=O)CCNc1ccc(C2=Nn3c(nnc3-c3ccccc3OCC)SC2)cc1]
or a pharmaceutically acceptable salt thereof.

8. A process of synthesizing the compound of claim 1, the process comprising: reacting a 4-amino-4H-1,2,4-triazole-3-thiol intermediate A with intermediate B wherein “LG” is a leaving group to afford the compound of Formula I

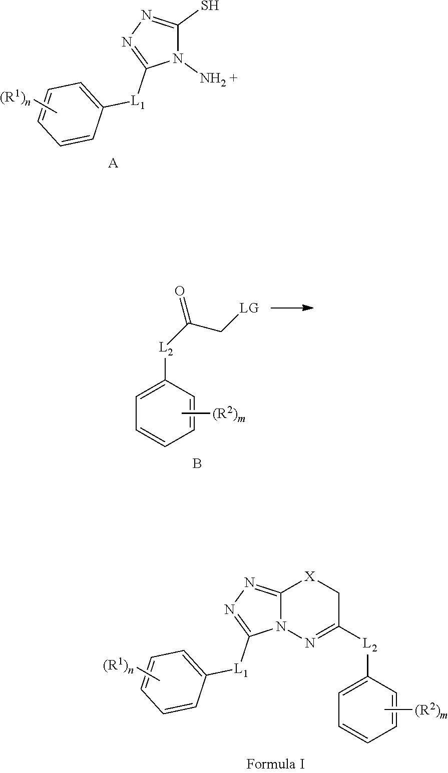

9. A method of treating a patient in need of treatment with a CXCR4 agonist, the method comprising
administering to the patient an effective amount of the compound of Formula I of any one of claims 1-7 or a compound of Formula II 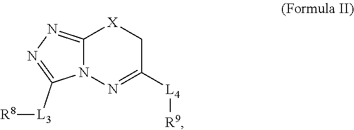
or a pharmaceutically acceptable salt thereof, wherein in Formula II,
X is CH₂, O, NRS, or S;
L₃ and L₄ are each independently a bond (absent) or —C₁-C₂₀hydrocarbyl;
R⁸ and R⁹ are each independently —C₃-C₇cycloalkyl, a heterocycloalkyl, a heteroaryl, or an aryl, each R⁸ and R⁹ is optionally substituted with halogen, cyano, nitro, oxo (C═O), —C₁-C₆alkyl, —C₂-C₆alkenyl, —C₂-C₆alkynyl, —C₁-C₆alkoxy, —C₀-C₆alkylCOR¹⁰, —C₀-C₆alkylCOOR¹⁰, —C₀-C₆alkyl(C₃-C₇cycloalkyl), —C₀-C₆alkyl(heterocycloalkyl), —C₀-C₆alkyl(aryl), or —C₀-C₆alkyl(heteroaryl), —NR¹⁰C₀-C₆alkylCOR¹¹, or NR¹⁰R¹¹, wherein groups except hydrogen, halogen, cyano, and nitro are optionally substituted with halogen, cyano, nitro, oxo, —C₁-C₆alkyl, —C₁-C₆haloalkyl, —C₃-C₇cycloalkyl, a heterocycloalkyl, a heteroaryl, or an aryl;
R¹⁰ and R¹¹ are each independently hydrogen, —C₁-C₆alkyl, —C₂-C₆alkenyl, —C₂-C₆alkynyl, —C₀-C₆alkyl-C₁-C₆alkoxy, —C₀-C₆alkylCOR¹², —C₀-C₆alkyl-COOR¹², —C₀-C₆alkyl-C₂-C₆alkenyl, C₀-C₆alkyl-C₂-C₆alkynyl, —C₀-C₆alkyl(aryl), or —C₀-C₆alkyl(heteroaryl), wherein groups except hydrogen are optionally substituted with halogen, cyano, nitro, oxo, —C₀-C₆alkylCOOR¹², —C₁-C₆alkyl, —C₁-C₆haloalkyl, —C₃-C₇cycloalkyl, a heterocycloalkyl, a heteroaryl, or an aryl;
or for NR¹⁰R¹¹, R¹⁰ and R¹¹ along with the N form a cyclic ring of 4 to 7 ring atoms or bicyclic heterocyclic ring of 8 to 10 ring atoms, the cyclic or bicyclic ring having 1, 2, or 3 ring atoms independently chosen from N, O, and S, wherein the cyclic or bicyclic heterocyclic ring is substituted with 0-2 substituents independently chosen from halogen, cyano, oxo, —C₁-C₆alkyl, —C₁-C₆alkoxy, —C₁-C₆haloalkyl, and —C₁-C₆haloalkoxy; and
R⁵ and R¹² are each independently hydrogen, —C₁-C₆alkyl, —C₁-C₆haloalkyl, —C₀-C₆alkyl(C₃-C₇cycloalkyl), —C₁-C₆alkyl-C₁-C₆alkoxy, —C₁-C₆alkyl-C₂-C₆alkenyl, or —C₁-C₆alkyl-C₂-C₆alkynyl.

10. The method of claim 9, wherein the patient is a diabetic patient.

11. The method of claim 9, wherein the patient is with a diabetic wound.

12. The method of claim 9, wherein the patient is with a decreased expression of stromal cell-derived factor-1α (SDF-1α).

13. The method of claim 9, wherein the compound of Formula II or a pharmaceutically acceptable salt thereof is a CXC chemokine receptor type 4 (CXCR4) agonist.

14. The method of claim 9, wherein the patient is a human.

15. The method of claim 9, comprising treating a wound.

16. The method of claim 15, comprising treating a diabetic wound.

17. The method of claim 9, comprising promoting wound healing.

18. The method of claim 17, comprising promoting diabetic wound healing.

19. A pharmaceutical formulation comprising a compound according to any one of claims 1-7 and a pharmaceutically acceptable carrier.

20. The pharmaceutical formulation of claim 19 formulated for topical or parenteral administration.

21. The method of claim 9, comprising administering to the patient an effective amount of one or more compound in Tables 2-4.

22. A method of treating a patient in need of treatment with a CXCR4 agonist, the method comprising administering to the patient an effective amount of the compound of Formula IIIa, wherein the compound is of Formula IIIa 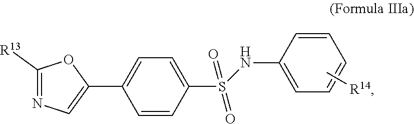
or a pharmaceutically acceptable salt thereof, wherein
R¹³ is hydrogen, halogen, cyano, nitro, —C₁-C₆alkyl, —C₂-C₆alkenyl, —C₂-C₆alkynyl, —C₁-C₆alkoxy, —C₀-C₆alkylCOR¹⁵, —C₀-C₆alkylCOOR¹⁵, —C₀-C₆alkyl(C₃-C₇cycloalkyl), —C₀-C₆alkyl(heterocycloalkyl), —C₀-C₆alkyl(aryl), or —C₀-C₆alkyl(heteroaryl), —NR¹⁶C₀-C₆alkylCOR¹⁵, or NR¹⁶R¹⁷, wherein groups except hydrogen, halogen, cyano, and nitro are optionally substituted with halogen, cyano, nitro, oxo, —C₁-C₆alkyl, —C₁-C₆haloalkyl, —C₃-C₇cycloalkyl, a heterocycloalkyl, a heteroaryl, or an aryl.

23. The method of claim 22, wherein the patient is a diabetic patient.

24. The method of claim 22, wherein the patient is with a diabetic wound.

25. The method of claim 22, wherein the patient is with a decreased expression of stromal cell-derived factor-1α (SDF-1α).

26. The method of claim 22, wherein the compound of Formula IIIa or a pharmaceutically acceptable salt thereof is a CXC chemokine receptor type 4 (CXCR4) agonist.

27. The method of claim 22, wherein the patient is a human.

28. The method of claim 22, comprising treating a wound.

29. The method of claim 28, comprising treating a diabetic wound.

30. The method of claim 22, comprising promoting wound healing.

31. The method of claim 30, comprising promoting diabetic wound healing.
